(19) United States
(12) Patent Application Publication
(10) Pub. No.: US 2026/0109692 A1
(43) Pub. Date: Apr. 23, 2026

(54) KCNT1 INHIBITORS AND METHODS OF USE

(71) Applicants: UCB BIOPHARMA SRL, Brussels (BE); Praxis Precision Medicines, Inc., Boston, MA (US)

(72) Inventors: James Thomas REUBERSON, Slough (GB); Sarah Jane FRITH, Nottingham (GB); Vijayendar Reddy YEDULLA, Nottingham (GB)

(21) Appl. No.: 19/361,128

(22) Filed: Oct. 17, 2025

(30) Foreign Application Priority Data
Oct. 18, 2024 (EP) 24207605.7

(51) Int. Cl. C07D 413/14; A61K 31/4439; C07B 59/00

(52) U.S. Cl. CPC C07D 413/14; A61K 31/4439; C07B 59/002

(57) ABSTRACT

The present invention is directed to, in part, compounds and compositions useful for preventing and/or treating a neurological disease or disorder, a disease or condition relating to excessive neuronal excitability, and/or a gain-of-function mutation in a gene (e.g., KCNT1). Methods of treating a neurological disease or disorder, a disease or condition relating to excessive neuronal excitability, and/or a gain-of-function mutation in a gene such as KCNT1 are also provided herein.

### CROSS-REFERENCE TO RELATED APPLICATIONS

This application claims the benefit of priority of European Patent Application No. EP24207605.7, filed Oct. 18, 2024, the disclosure of which is incorporated herein by reference in its entirety.

### BACKGROUND

KCNT1 encodes sodium-activated potassium channels known as Slack (Sequence like a calcium-activated K⁺ channel). These channels are found in neurons throughout the brain and can mediate a sodium-activated potassium current I_{KNa}. This delayed outward current can regulate neuronal excitability and the rate of adaption in response to maintained stimulation. Abnormal Slack activity has been associated with development of early onset epilepsies and intellectual impairment. Accordingly, pharmaceutical compounds that selectively regulate sodium-activated potassium channels, e.g., abnormal KCNT1, abnormal I_{KNa}, are useful in treating a neurological disease or disorder or a disease or condition related to excessive neuronal excitability and/or KCNT1 gain-of-function mutations.

WO2020/227101 and WO2021/173930, for example, describe other pharmaceutical compounds useful in regulating sodium-activated potassium channels.

### SUMMARY OF THE INVENTION

Described herein are compounds and pharmaceutical compositions useful for preventing and/or treating a disease, disorder, or condition, e.g., a neurological disease or disorder, a disease, disorder, or condition associated with excessive neuronal excitability and/or a gain-of-function mutation in KCNT1.

The compounds of the present invention may provide potent inhibition of KCNT1, low levels of metabolic clearance, high in vivo stability and good solubility in biological media, for example media relevant for oral administration, and intestinal permeability.

In one aspect, the present invention provides a compound of Formula (I):

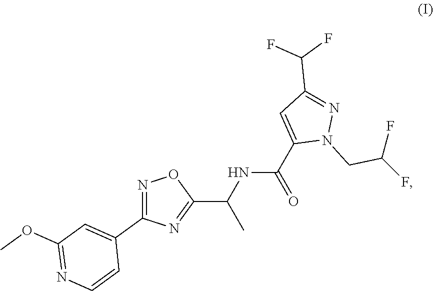 [SMILES: COc1cc(-c2noc(C(C)NC(=O)c3cc(C(F)F)nn3CC(F)F)n2)ccn1]

or a salt, solvate and/or isotopologue thereof.

The compound of formula (I) may be provided in the form of a salt, solvate and/or isotopologue thereof. The compound of formula (I) may be provided in the form of a salt thereof. The compound of formula (I) may be provided in the form of a solvate thereof. The compound of formula (I) may be provided in the form of an isotopologue thereof. The compound of formula (I) may be provided in the form of a pharmaceutically acceptable salt, solvate and/or isotopologue thereof. The compound of formula (I) may be provided in the form of a pharmaceutically acceptable salt thereof. The compound of formula (I) may be provided in the form of a pharmaceutically acceptable solvate thereof. The compound of formula (I) may be provided as a free form, for example in the form of the free base.

There is no specific disclosure in WO2020/227101 or WO2021/173930 of a compound of formula (I) as defined above, or a salt, solvate and/or isotopologue thereof.

In a particular embodiment according to this aspect, the present invention provides a compound of Formula (II):

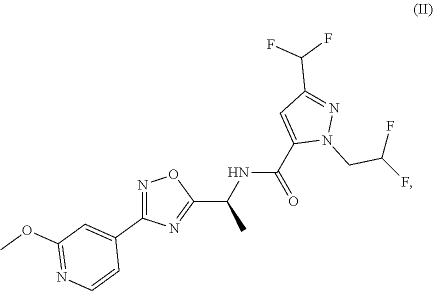 [SMILES: COc1cc(-c2noc([C@H](C)NC(=O)c3cc(C(F)F)nn3CC(F)F)n2)ccn1]

or a salt, solvate and/or isotopologue thereof.

It will be appreciated that the compound of formulae (II) is a sub-formulae of the compound of formula (I). Any reference to a compound of formula (I) contained herein, will therefore include the compound of formula (II).

In another aspect, the present invention provides a pharmaceutical composition comprising a compound disclosed herein, or a pharmaceutically acceptable salt, solvate or isotopologue thereof, and a pharmaceutically acceptable excipient.

In another aspect, the present invention provides a compound disclosed herein, or a pharmaceutically acceptable salt, solvate and/or isotopologue thereof, or a pharmaceutical composition disclosed herein, for use in therapy.

In another aspect, the present invention provides a method of treating a neurological disease or disorder, wherein the method comprises administering to a subject in need thereof a compound disclosed herein, or a pharmaceutically acceptable salt, solvate and/or isotopologue thereof, or a pharmaceutical composition disclosed herein.

In another aspect, the present invention provides a method of treating a disease or condition associated with excessive neuronal excitability, wherein the method comprises administering to a subject in need thereof a compound disclosed herein, or a pharmaceutically acceptable salt, solvate and/or isotopologue thereof, or a pharmaceutical composition disclosed herein.

In another aspect, the present invention provides a method of treating a disease or condition associated with a gain-of-function mutation of KCNT1, wherein the method comprises administering to a subject in need thereof a compound disclosed herein, or a pharmaceutically acceptable salt, solvate and/or isotopologue thereof, or a pharmaceutical composition disclosed herein.

In another aspect, the present invention provides a compound disclosed herein, or a pharmaceutically acceptable salt, solvate and/or isotopologue thereof, or a pharmaceutical composition disclosed herein for use in treating a neurological disease or disorder.

In another aspect, the present invention provides a compound disclosed herein, or a pharmaceutically acceptable salt, solvate and/or isotopologue thereof, or a pharmaceutical composition disclosed herein for use in treating a disease or condition associated with excessive neuronal excitability.

In another aspect, the present invention provides a compound disclosed herein, or a pharmaceutically acceptable salt, solvate and/or isotopologue thereof, or a pharmaceutical composition disclosed herein for use in treating a disease or condition associated with a gain-of-function mutation of KCNT1.

In another aspect, the present invention provides the use of a compound disclosed herein, or a pharmaceutically acceptable salt, solvate and/or isotopologue thereof, or a pharmaceutical composition disclosed herein in the manufacture of a medicament for the treatment of a neurological disease or disorder.

In another aspect, the present invention provides the use of a compound disclosed herein, or a pharmaceutically acceptable salt, solvate and/or isotopologue thereof, or a pharmaceutical composition disclosed herein in the manufacture of a medicament for the treatment of a disease or condition associated with excessive neuronal excitability.

In another aspect, the present invention provides the use of a compound disclosed herein, or a pharmaceutically acceptable salt, solvate and/or isotopologue thereof, or a pharmaceutical composition disclosed herein in the manufacture of a medicament for the treatment of a disease or condition associated with a gain-of-function mutation of KCNT1.

The neurological disease or disorder, the disease or condition associated with excessive neuronal excitability, or the disease or condition associated with a gain-of-function mutation of a KCNT1, may be a KCNT-1 related epilepsy or neurodevelopmental disorder.

The neurological disease or disorder, the disease or condition associated with excessive neuronal excitability, or the disease or condition associated with a gain-of-function mutation of KCNT1 may be epilepsy, an epilepsy syndrome, or an encephalopathy.

The neurological disease or disorder, the disease or condition associated with excessive neuronal excitability, or the disease or condition associated with a gain-of-function mutation of KCNT1 may be a genetic or pediatric epilepsy, a genetic or pediatric epilepsy syndrome, or a genetic or pediatric developmental encephalopathy.

The neurological disease or disorder, the disease or condition associated with excessive neuronal excitability, or the disease or condition associated with a gain-of-function mutation of KCNT1 may be a cardiac dysfunction.

The neurological disease or disorder, the disease or condition associated with excessive neuronal excitability, or the disease or condition associated with a gain-of-function mutation of KCNT1 may be selected from epilepsy and other encephalopathies. The epilepsy and other encephalopathies may be selected from epilepsy of infancy with migrating focal seizures (MMFSI, EIMFS), autosomal dominant nocturnal frontal lobe epilepsy (ADNFLE), West syndrome, infantile spasms, epileptic encephalopathy, focal epilepsy, Ohtahara syndrome, developmental and epileptic encephalopathy, Lennox Gastaut syndrome, Dravet Syndrome, seizures (e.g., Generalized tonic clonic seizures, Asymmetric Tonic Seizures), leukodystrophy, leukoencephalopathy, intellectual disability, Multifocal Epilepsy, Drug resistant epilepsy, Temporal lobe epilepsy, cerebellar ataxia.

The neurological disease or disorder, the disease or condition associated with excessive neuronal excitability, or the disease or condition associated with a gain-of-function mutation of KCNT1 may be selected from the group consisting of cardiac arrhythmia, sudden unexpected death in epilepsy, Brugada syndrome, and myocardial infarction.

The neurological disease or disorder, the disease or condition associated with excessive neuronal excitability, or the disease or condition associated with a gain-of-function mutation of KCNT1 may be selected from pain and related conditions (e.g. neuropathic pain, acute/chronic pain, migraine, etc).

The neurological disease or disorder, the disease or condition associated with excessive neuronal excitability, or the disease or condition associated with a gain-of-function mutation of KCNT1 may be a muscle disorder (e.g. myotonia, neuromyotonia, cramp muscle spasms, spasticity).

The neurological disease or disorder, the disease or condition associated with excessive neuronal excitability, or the disease or condition associated with a gain-of-function mutation of KCNT1 may be selected from itch and pruritis, ataxia and cerebellar ataxias.

The neurological disease or disorder, the disease or condition associated with excessive neuronal excitability, or the disease or condition associated with a gain-of-function mutation of KCNT1 may be selected from psychiatric disorders (e.g. major depression, anxiety, bipolar disorder, schizophrenia).

The neurological disease or disorder or the disease or condition associated with excessive neuronal excitability and/or a gain-of-function mutation in KCNT1 may be selected from the group consisting of learning disorders, Fragile X, neuronal plasticity, and autism spectrum disorders.

The neurological disease or disorder, the disease or condition associated with excessive neuronal excitability, or the disease or condition associated with a gain-of-function mutation of a KCNT1, may be selected from the group consisting of epileptic encephalopathy with SCN1A, SCN2A, SCN8A mutations, early infantile epileptic encephalopathy, Dravet syndrome, Dravet syndrome with SCN1A mutation, generalized epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, sudden unexpected death in epilepsy, Rasmussen encephalitis, malignant migrating partial seizures of infancy, autosomal dominant nocturnal frontal lobe epilepsy, sudden unexpected death in epilepsy (SUDEP), KCNQ2 epileptic encephalopathy, and KCNT1 epileptic.

Other objects and advantages will become apparent to those skilled in the art from a consideration of the ensuing Detailed Description, Examples, and Claims.

### DETAILED DESCRIPTION OF THE INVENTION

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Formula (I) and the formulae depicted hereinafter are intended to represent all individual stereoisomers and all possible mixtures thereof, unless stated or shown otherwise.

The carbon-carbon bonds of the compound of formula (I) are depicted herein using a solid line ( [symbol] ), a solid wedge ( [symbol] ), or a dotted wedge ( [symbol] ). The use of a solid line to depict bonds to asymmetric carbon atoms is meant to indicate that all possible stereoisomers (e.g., specific enantiomers, racemic mixtures, etc.) at that carbon atom are included. The use of either a solid or dotted wedge to depict bonds to asymmetric carbon atoms is used to indicate the stereoisomer as shown. It is possible that the compound of formula (I) may contain more than one asymmetric carbon atom. In those compounds, the use of a solid line to depict bonds to asymmetric carbon atoms indicates that all possible stereoisomers are included.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. The weights may be based upon total weight of all enantiomers or stereoisomers of the compound.

It is also to be understood that each individual atom present in formula (I), or in the formulae depicted hereinafter, may be present in the form of any of its naturally occurring isotopes. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; F may be in any isotopic form, including ¹⁸F and ¹⁹F; and the like. Thus, the present invention, also includes within its scope, isotopically-labelled compounds of Formula (I) referred to herein as isotopologues.

The following terms are intended to have the meanings presented therewith below and are useful in understanding the description and intended scope of the present invention. When describing the invention, which may include compounds and pharmaceutically acceptable salts, solvates and/or isotopologues thereof, pharmaceutical compositions containing such compounds and methods of using such compounds and compositions, the following terms, if present, have the following meanings unless otherwise indicated. The articles “a” and “an” may be used herein to refer to one or to more than one (i.e. at least one) of the grammatical objects of the article. By way of example “an analogue” means one analogue or more than one analogue.

When a range of values is listed, it is intended to encompass each value and sub-range within the range.

These and other exemplary substituents are described in more detail in the Examples and Claims. The invention is not intended to be limited in any manner by the above exemplary listing of substituents.

### Other Definitions

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19, and Gould, Salt selection for basic drugs, International Journal of Pharmaceutics, 33 (1986) 201-217. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. The subject may be a human. The subject may be a non-human animal.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (also “therapeutic treatment”).

In general, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

The present invention also contemplates administration of the compounds, or pharmaceutically acceptable salts, solvates or isotopologues thereof, of the present invention, or a pharmaceutical composition of the present invention, as a prophylactic before a subject begins to suffer from the specified disease, disorder or condition. As used herein, “prophylactic treatment” contemplates an action that occurs before a subject begins to suffer from the specified disease, disorder or condition. As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

As used herein, a “disease or condition associated with a gain-of-function mutation of KCNT1” refers to a disease or condition that is associated with, is partially or completely caused by, or has one or more symptoms that are partially or completely caused by, a mutation in KCNT1 that results in a gain-of-function phenotype, i.e. an increase in activity of the potassium channel encoded by KCNT1 resulting in an increase in whole cell current. Activity can be assessed by, for example, ion flux assay or electrophysiology (e.g. using the whole cell patch clamp technique). Typically, a gain-of-function mutation results in an increase of at least or about 20%, 30%, 40%, 50%, 60%, 70%, 80%, 90%, 100%, 125%, 150%, 175%, 200%, 225%, 250%, 275%, 300%, 325%, 350%, 375%, 400% or more compared to the activity of a potassium channel encoded by a wild-type KCNT1.

### Compounds

In one aspect, the present invention provides a compound of Formula (I):

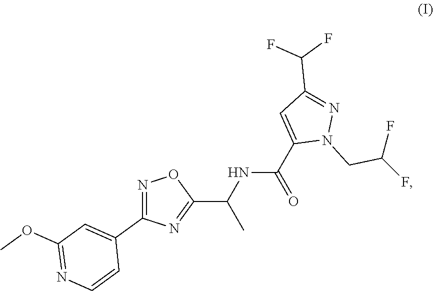 [SMILES: COc1cc(-c2noc(C(C)NC(=O)c3cc(C(F)F)nn3CC(F)F)n2)ccn1]

which is 2-(2,2-Difluoroethyl)-5-(difluoromethyl)-N-[1-[3-(2-methoxy-4-pyridyl)-1,2,4-oxadiazol-5-yl]ethyl]pyrazole-3-carboxamide, or a salt, solvate, and/or isotopologue thereof.

In a particular embodiment according to this aspect, the present invention provides a compound of Formula (II):

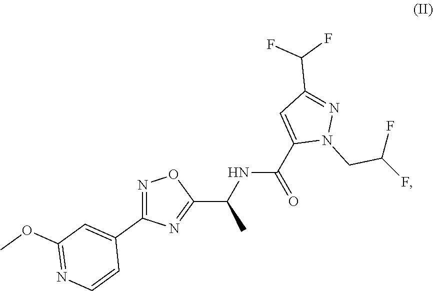 [SMILES: COc1cc(-c2noc([C@H](C)NC(=O)c3cc(C(F)F)nn3CC(F)F)n2)ccn1]

which is 2-(2,2-Difluoroethyl)-5-(difluoromethyl)-N-[(1S)-1-[3-(2-methoxy-4-pyridyl)-1,2,4-oxadiazol-5-yl]ethyl]pyrazole-3-carboxamide, or a salt, solvate and/or isotopologue thereof.

The present invention further provides a salt of a compound of formula (I) or (II). The present invention further provides a pharmaceutically acceptable salt of a compound of formula (I) or (II). The present invention further provides a solvate of a compound of formula (I) or (II). The present invention further provides a pharmaceutically acceptable solvate of a compound of formula (I) or (II). The present invention further provides a pharmaceutically acceptable salt and/or solvate of a compound of formula (I) or (II). The present invention further provides an isotopologue of a compound of formula (I) or (II). The present invention further provides a compound of formula (I) or (II) in free form, for example as the free base.

The present invention includes within its scope tautomers of compounds of formula (I) or (II), for example an amide (NHC═O)↔hydroxyimine (N═COH) tautomer. Formula (I) and the formulae depicted hereinafter are intended to represent all individual tautomers and all possible mixtures thereof, unless stated or shown otherwise.

The present invention includes within its scope solvates of the compound of formula (I) or (II) above. Such solvates may be formed with common organic solvents or water (for example, hydrates).

The present invention also includes within its scope co-crystals of the compound of formula (I) or (II) above. The technical term “co-crystal” is used to describe the situation where neutral molecular components are present within a crystalline compound in a definite stoichiometric ratio. The preparation of pharmaceutical co-crystals enables modifications to be made to the crystalline form of an active pharmaceutical ingredient, which in turn can alter its physicochemical properties without compromising its intended biological activity (see Pharmaceutical Salts and Co-crystals, ed. J. Wouters & L. Quere, RSC Publishing, 2012).

The present invention also includes within its scope pro-drug forms of the compound of formula (I) or (II) and its various sub-scopes and sub-groups.

The present invention is therefore intended to encompass the compounds disclosed herein, and the pharmaceutically acceptable salts, solvates, isotopologues, tautomeric forms, and prodrugs of such compounds.

In one particular embodiment according to the present invention, the compound of formula (I) may be:

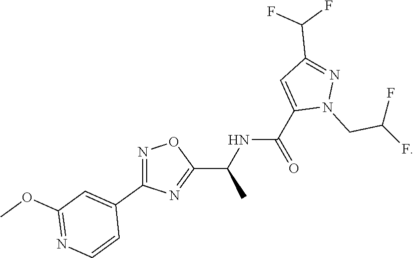 [SMILES: COc1cc(-c2noc([C@H](C)NC(=O)c3cc(C(F)F)nn3CC(F)F)n2)ccn1]

In other words, the compound may be in the free base form, not a salt form.

In another particular embodiment according to the present invention, the compound of formula (I) may be a salt of:

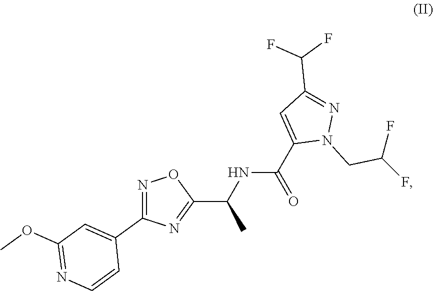 [SMILES: COc1cc(-c2noc([C@H](C)NC(=O)c3cc(C(F)F)nn3CC(F)F)n2)ccn1]

In another particular embodiment according to the present invention, the compound of formula (I) may be a pharmaceutically acceptable salt of:

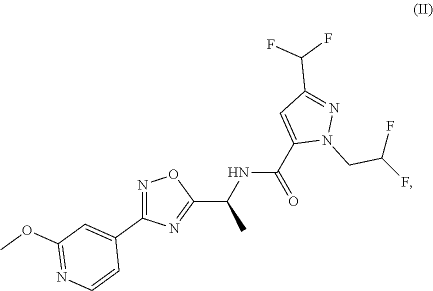 [SMILES: COc1cc(-c2noc([C@H](C)NC(=O)c3cc(C(F)F)nn3CC(F)F)n2)ccn1]

In another particular embodiment according to the present invention, the compound of formula (I) may be an isotopologue of:

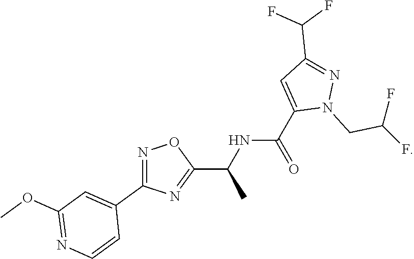 [SMILES: COc1cc(-c2noc([C@H](C)NC(=O)c3cc(C(F)F)nn3CC(F)F)n2)ccn1]

An isotopologue of the compound of formula (I) or (II) may comprise at least one isotopic substitution, for example, 1, 2 or 3 isotopic substitutions. The isotopologue may comprise 3 isotopic substitutions.

For example, at least one H atom may be substituted for D. For example, one, two, or three H atoms may be substituted for D. Three H atoms may be substituted for D.

In one particular embodiment, an isotopologue may be:

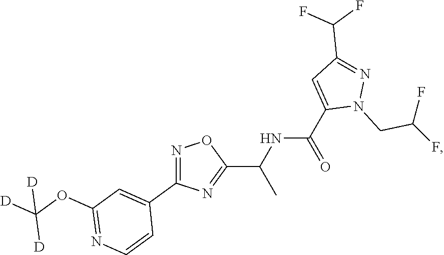 [SMILES: [2H]C([2H])([2H])Oc1cc(-c2noc(C(C)NC(=O)c3cc(C(F)F)nn3CC(F)F)n2)ccn1]

which is 2-(2,2-Difluoroethyl)-5-(difluoromethyl)-N-[1-[3-[2-(trideuteriomethoxy)-4-pyridyl]-1,2,4-oxadiazol-5-yl]ethyl]pyrazole-3-carboxamide, or a salt and/or solvate thereof.

In another particular embodiment, an isotopologue may be:

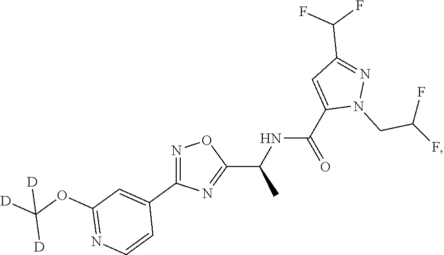 [SMILES: [2H]C([2H])([2H])Oc1cc(-c2noc([C@H](C)NC(=O)c3cc(C(F)F)nn3CC(F)F)n2)ccn1]

which is 2-(2,2-Difluoroethyl)-5-(difluoromethyl)-N-[(1S)-1-[3-[2-(trideuteriomethoxy)-4-pyridyl]-1,2,4-oxadiazol-5-yl]ethyl]pyrazole-3-carboxamide;
or a salt and/or solvate thereof.

Isotopologues of the compounds according to the present invention may be provided in the form of a salt and/or solvate thereof. Isotopologues of the compounds may be provided in the form of a salt thereof. Isotopologues of the compounds may be provided in the form of a solvate thereof. Isotopologues of the compounds may be provided in the form of a pharmaceutically acceptable salt and/or solvate thereof. Isotopologues of the compounds may be provided in the form of a pharmaceutically acceptable salt thereof. Isotologues of the compounds may be provided in the form of a pharmaceutically acceptable solvate thereof. Isotologues of the compounds may be provided in free form, for example in the form of the free base.

### General Synthetic Schemes

Exemplary methods for preparing compounds described herein are illustrated in the following synthetic schemes. These schemes are given for the purpose of illustrating the invention, and should not be regarded in any manner as limiting the scope of the invention.

The synthetic route illustrated in Scheme 1 depicts an exemplary procedure for preparing carboxylic acid intermediate D. In the first step, compound A is reacted with 2,2-difluoroethyl trifluoromethanesulfonate in the presence of a base to form ethyl pyrazole-5-carboxylate C. Then, hydrolysis of C provides carboxylic acid D.

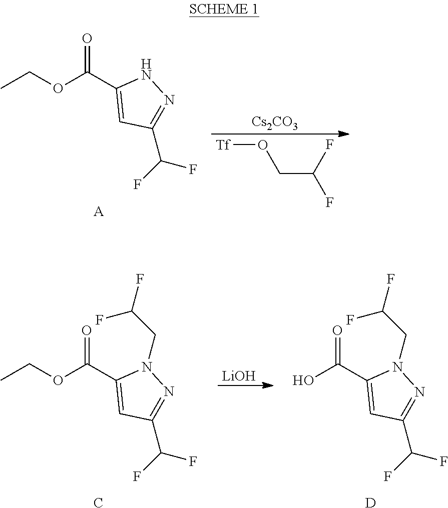

The synthetic route illustrated in Scheme 2 depicts an exemplary procedure for preparing amine-substituted oxadiazole intermediate L. In the first step, nitrile H is treated with hydroxylamine to provide N-hydroxyimidamide I. Then, cabonyldiimidazole (CDI)-mediated cyclization of I with Boc-L-alanine affords oxadiazole K. Deprotection of K under acidic conditions, followed by neutralisation, provides amine-substituted oxadiazole intermediate L.

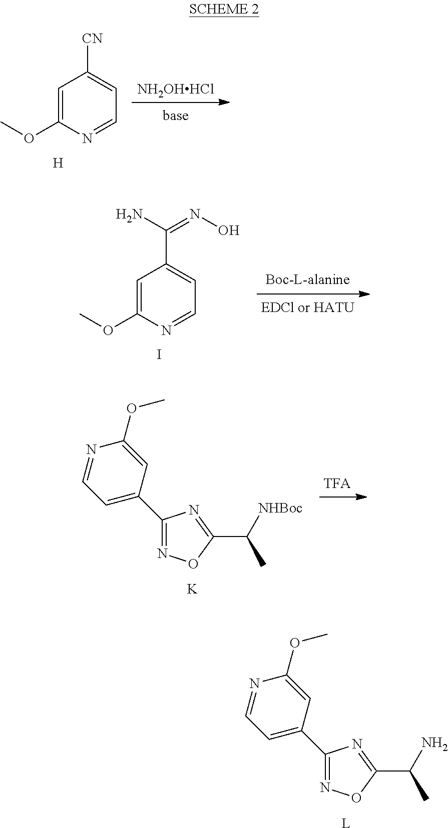

The synthetic route illustrated in Scheme 3 depicts an exemplary procedure for preparing M (a compound of formula (I)). Coupling of amine-substituted oxadiazole intermediate L with carboxylic acid D using standard peptide coupling procedures (e.g., HOBt, and EDCI in dichloromethane in the presence of DIPEA, or HATU in DMF in the presence of DIPEA) provides compound M (a compound of formula (I)).

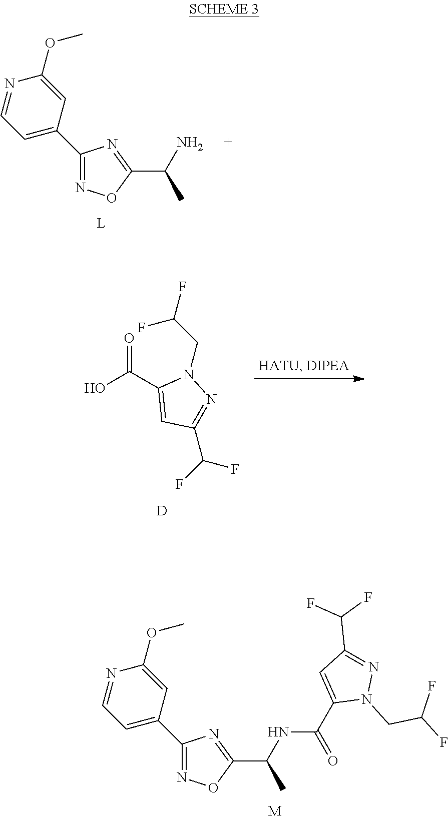

Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The invention additionally encompasses compounds described herein as individual isomers substantially free of other isomers, and alternatively, as mixtures of various isomers.

Isotopologues of the compounds of the present invention may be prepared from corresponding isotopically labelled reagents using the methods described above and herein.

### Methods of Treatment

The compounds, or pharmaceutically acceptable salts, solvates or isotopologues thereof, and compositions described above and herein can be used to treat a neurological disease or disorder. The compounds, or pharmaceutically acceptable salts, solvates or isotopologues thereof, and compositions described above and herein can be used to treat a disease or condition associated with excessive neuronal excitability. The compounds, or pharmaceutically acceptable salts, solvates or isotopologues thereof, and compositions described above and herein can also be used to treat a disease or condition associated with a gain-of-function mutation in KCNT1. Exemplary diseases, disorders, or conditions that can be treated with the compounds and compositions described above and herein include epilepsy and other encephalopathies (e.g., epilepsy of infancy with migrating focal seizures (MMFSI, EIMFS), autosomal dominant nocturnal frontal lobe epilepsy (ADNFLE), West syndrome, infantile spasms, epileptic encephalopathy, developmental and epileptic encephalopathy (DEE), early infantile epileptic encephalopathy (EIEE), generalized epilepsy, focal epilepsy, multifocal epilepsy, temporal lobe epilepsy, Ohtahara syndrome, early myoclonic encephalopathy and Lennox Gastaut syndrome, drug resistant epilepsy, seizures (e.g., frontal lobe seizures, generalized tonic clonic seizures, asymmetric tonic seizures, focal seizures), leukodystrophy, hypomyelinating leukodystrophy, leukoencephalopathy, sudden unexpected death in epilepsy, epileptic encephalopathy with SCN1A, SCN2A, SCN8A mutations, early infantile epileptic encephalopathy, Dravet syndrome, Dravet syndrome with SCN1A mutation, generalized epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, Rasmussen encephalitis, malignant migrating partial seizures of infancy, autosomal dominant nocturnal frontal lobe epilepsy, sudden unexpected death in epilepsy (SUDEP), KCNQ2 epileptic encephalopathy, KCNT1 epileptic encephalopathy, a genetic or pediatric epilepsy, a genetic or pediatric epilepsy syndrome, a genetic or pediatric developmental encephalopathy, early-onset epilepsy (EOE), and early-onset epileptic encephalopathy (EOEE)), cardiac dysfunctions (e.g., cardiac arrhythmia, Brugada syndrome, myocardial infarction), pulmonary vasculopathy/hemorrhage, pain and related conditions (e.g. neuropathic pain, acute/chronic pain, migraine, etc), muscle disorders (e.g. myotonia, neuromyotonia, cramp muscle spasms, spasticity), itch and pruritis, movement disorders (e.g., ataxia and cerebellar ataxias), psychiatric disorders (e.g. major depression, anxiety, bipolar disorder, schizophrenia, attention-deficit hyperactivity disorder), neurodevelopmental disorder, learning disorders, intellectual disability, Fragile X, neuronal plasticity, and autism spectrum disorders.

The neurological disease or disorder or the disease or condition associated with excessive neuronal excitability and/or a gain-of-function mutation in KCNT1 may be selected from EIMFS, ADNFLE and West syndrome. The neurological disease or disorder or the disease or condition associated with excessive neuronal excitability and/or a gain-of-function mutation in KCNT1 may be selected from infantile spasms, epileptic encephalopathy, focal epilepsy, Ohtahara syndrome, developmental and epileptic encephalopathy, Lennox Gastaut syndrome, and Dravet syndrome. The neurological disease or disorder or the disease or condition associated with excessive neuronal excitability and/or a gain-of-function mutation in KCNT1 may be seizure. The neurological disease or disorder or the disease or condition associated with excessive neuronal excitability and/or a gain-of-function mutation in KCNT1 may be selected from a genetic or pediatric epilepsy, or a genetic or pediatric epilepsy syndrome, or a genetic or pediatric developmental encephalopathy. The neurological disease or disorder or the disease or condition associated with excessive neuronal excitability and/or a gain-of-function mutation in KCNT1 may be early-onset epilepsy (EOE) or early-onset epileptic encephalopathy (EOEE). The neurological disease or disorder or the disease or condition associated with excessive neuronal excitability and/or a gain-of-function mutation in KCNT1 may be selected from cardiac arrhythmia, Brugada syndrome, and myocardial infarction.

The neurological disease or disorder or the disease or condition associated with excessive neuronal excitability and/or a gain-of-function mutation in KCNT1 may be selected from the group consisting of the learning disorders, Fragile X, intellectual disability, neuronal plasticity, psychiatric disorders, and autism spectrum disorders.

The neurological disease or disorder or the disease or condition associated with excessive neuronal excitability and/or a gain-of-function mutation in KCNT1 may be selected from the group consisting of epileptic encephalopathy with SCN1A, SCN2A, SCN8A mutations, early infantile epileptic encephalopathy, Dravet syndrome, Dravet syndrome with SCN1A mutation, generalized epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, sudden unexpected death in epilepsy, Rasmussen encephalitis, malignant migrating partial seizures of infancy, autosomal dominant nocturnal frontal lobe epilepsy, sudden unexpected death in epilepsy (SUDEP), KCNQ2 epileptic encephalopathy, and KCNT1 epileptic encephalopathy.

Accordingly, the compounds disclosed herein, or pharmaceutically acceptable salts, solvates or isotopologues thereof, and pharmaceutical compositions thereof can be administered to a subject with a neurological disease or disorder or a disease or condition associated with excessive neuronal excitability and/or a gain-of-function mutation in KCNT1 (e.g., EIMFS, ADNFLE, West syndrome, infantile spasms, epileptic encephalopathy, focal epilepsy, Ohtahara syndrome, developmental and epileptic encephalopathy, Lennox Gastaut syndrome, Dravet syndrome, seizures, cardiac arrhythmia, Brugada syndrome, and myocardial infarction).

EIMFS is a rare and debilitating genetic condition characterized by an early onset (before 6 months of age) of almost continuous heterogeneous focal seizures, where seizures appear to migrate from one brain region and hemisphere to another. Patients with EIMFS are generally intellectually impaired, non-verbal and non-ambulatory. While several genes have been implicated to date, the gene that is most commonly associated with EIMFS is KCNT1. Several de novo mutations in KCNT1 have been identified in patients with EIMFS, including but not limited to V271F, G288S, R428Q, R474Q, R474H, R474C, I760M, A934T, P924L, G243S, H257D, A259D, R262Q, Q270E, L274I, F346L, C377S, R398Q, P409S, A477T, F502V, M516V, Q550del, K629E, K629N, I760F, E893K, M896K, R933G, R950Q, K1154Q (Barcia et al. (2012) Nat Genet. 44: 1255-1260; Ishii et al. (2013) Gene 531:467-471; McTague et al. (2013) Brain. 136: 1578-1591; Epi4K Consortium & Epilepsy Phenome/Genome Project. (2013) Nature 501:217-221; Lim et al. (2016) Neurogenetics; Ohba et al. (2015) Epilepsia 56:e121-e128; Zhou et al. (2018) Genes Brain Behav. e12456; Moller et al. (2015) Epilepsia. e114-20; Numis et al. (2018) Epilepsia. 1889-1898; Madaan et al. Brain Dev. 40(3):229-232; McTague et al. (2018) Neurology. 90(1):e55-e66; Kawasaki et al. (2017) J Pediatr. 191:270-274; Kim et al. (2014) Cell Rep. 9(5):1661-1672; Ohba et al. (2015) Epilepsia. 56(9):e121-8; Rizzo et al. (2016) Mol Cell Neurosci. 72:54-63; Zhang et al. (2017) Clin Genet. 91(5):717-724; Mikati et al. (2015) Ann Neurol. 78(6):995-9; Baumer et al. (2017) Neurology. 89(21):2212; Dilena et al. (2018) Neurotherapeutics. 15(4):1112-1126). These mutations are gain-of-function, missense mutations that are dominant (i.e. present on only one allele) and result in change in function of the encoded potassium channel that causes a marked increase in whole cell current when tested in Xenopus oocyte or mammalian expression systems (see e.g. Milligan et al. (2015) Ann Neurol. 75(4): 581-590; Barcia et al. (2012) Nat Genet. 44(11): 1255-1259; and Mikati et al. (2015) Ann Neurol. 78(6): 995-999).

ADNFLE has a later onset than EIMFS, generally in mid-childhood, and is generally a less severe condition. It is characterized by nocturnal frontal lobe seizures and can result in psychiatric, behavioural and cognitive disabilities in patients with the condition. While ADNFLE is associated with genes encoding several neuronal nicotinic acetylcholine receptor subunits, mutations in the KCNT1 gene have been implicated in more severe cases of the disease (Heron et al. (2012) Nat Genet. 44: 1188-1190). Functional studies of the mutated KCNT1 genes associated with ADNFLE indicated that the underlying mutations (including but not limited to M896I, R398Q, Y796H and R928C) were dominant, gain-of-function mutations (Milligan et al. (2015) Ann Neurol. 75(4): 581-590; Mikati et al. (2015) Ann Neurol. 78(6): 995-999).

West syndrome is a severe form of epilepsy composed of a triad of infantile spasms, an interictal electroencephalogram (EEG) pattern termed hypsarrhythmia, and mental retardation, although a diagnosis can be made one of these elements is missing. Mutations in KCNT1, including but not limited to G652V and R474H, have been associated with West syndrome (Fukuoka et al. (2017) Brain Dev 39:80-83 and Ohba et al. (2015) Epilepsia 56:e121-e128). Treatment targeting the KCNT1 channel suggests that these mutations are gain-of-function mutations (Fukuoka et al. (2017) Brain Dev 39:80-83).

In one aspect, the present invention features a method of treating a disease or condition associated with excessive neuronal excitability and/or a gain-of-function mutation in KCNT1 (for example, epilepsy and other encephalopathies (e.g., epilepsy of infancy with migrating focal seizures (MMFSI, EIMFS), autosomal dominant nocturnal frontal lobe epilepsy (ADNFLE), West syndrome, infantile spasms, epileptic encephalopathy, focal epilepsy, Ohtahara syndrome, developmental and epileptic encephalopathy (DEE), and Lennox Gastaut syndrome, Dravet syndrome, seizures, leukodystrophy, leukoencephalopathy, intellectual disability, Multifocal Epilepsy, Generalized tonic clonic seizures, Drug resistant epilepsy, Temporal lobe epilepsy, cerebellar ataxia, Asymmetric Tonic Seizures) and cardiac dysfunctions (e.g., cardiac arrhythmia, Brugada syndrome, sudden unexpected death in epilepsy, myocardial infarction), pain and related conditions (e.g. neuropathic pain, acute/chronic pain, migraine, etc), muscle disorders (e.g. myotonia, neuromyotonia, cramp muscle spasms, spasticity), itch and pruritis, ataxia and cerebellar ataxias, psychiatric disorders (e.g. major depression, anxiety, bipolar disorder, schizophrenia), learning disorders, Fragile X, neuronal plasticity, and autism spectrum disorders) comprising administering to a subject in need thereof a compound disclosed herein or a pharmaceutically acceptable salt, solvate or isotopologue thereof, or a pharmaceutical composition disclosed herein.

In some examples, the subject presenting with a disease or condition that may be associated with a gain-of-function mutation in KCNT1 is genotyped to confirm the presence of a known gain-of-function mutation in KCNT1 prior to administration of the compounds, or pharmaceutically acceptable salts, solvates or isotopologues thereof or pharmaceutical compositions disclosed herein. For example, whole exome sequencing can be performed on the subject. Gain-of-function mutations associated with E IFS may include, but are not limited to, V271F, G288S, R428Q, R474Q, R474H, R474C, I760M, A934T, P924L, G243S, H257D, A259D, R262Q, Q270E, L274I, F346L, C377S, R398Q, P409S, A477T, F502V, M516V, Q550del, K629E, K629N, I760F, E893K, M896K, R933G, R950Q, and K1154Q. Gain-of-function mutations associated with ADNFLE may include, but are not limited to, M896I, R398Q, Y796H, R928C, and G288S. Gain-of-function mutations associated with West syndrome may include, but are not limited to, G652V and R474H. Gain-of-function mutations associated with temporal lobe epilepsy may include, but are not limited to, R133H and R565H. Gain-of-function mutations associated with Lennox-Gastaut may include, but are not limited to, R209C. Gain-of-function mutations associated with seizures may include, but are not limited to, A259D, G288S, R474C, R474H. Gain-of-function mutations associated with leukodystrophy may include, but are not limited to, G288S and Q906H. Gain-of-function mutations associated with Multifocal Epilepsy may include, but are not limited to, V340M. Gain-of-function mutations associated with EOE may include, but are not limited to, F346L and A934T. Gain-of-function mutations associated with Early-onset epileptic encephalopathies (EOEE) may include, but are not limited to, R428Q. Gain-of-function mutations associated with developmental and epileptic encephalopathies may include, but are not limited to, F346L, R474H, and A934T. Gain-of-function mutations associated with epileptic encephalopathies may include, but are not limited to, L437F, Y796H, P924L, R961H. Gain-of-function mutations associated with Early Infantile Epileptic Encephalopathy (EIEE) may include, but are not limited to, M896K. Gain-of-function mutations associated with drug resistant epilepsy and generalized tonic-clonic seizure may include, but are not limited to, F346L. Gain-of-function mutations associated with migrating partial seizures of infancy may include, but are not limited to, R428Q. Gain-of-function mutations associated with Leukoencephalopathy may include, but are not limited to, F932I. Gain-of-function mutations associated with NFLE may include, but are not limited to, A934T and R950Q. Gain-of-function mutations associated with Ohtahara syndrome may include, but are not limited to, A966T. Gain-of-function mutations associated with infantile spasms may include, but are not limited to, P924L. Gain-of-function mutations associated with Brugada Syndrome may include, but are not limited to, Ri 106Q. Gain-of-function mutations associated with Brugada Syndrome may include, but are not limited to, R474H.

In other examples, the subject is first genotyped to identify the presence of a mutation in KCNT1 and this mutation is then confirmed to be a gain-of-function mutation using standard in vitro assays, such as those described in Milligan et al. (2015) Ann Neurol. 75(4): 581-590. Typically, the presence of a gain-of-function mutation is confirmed when the expression of the mutated KCNT1 allele results an increase in whole cell current compared to the whole cell current resulting from expression of wild-type KCNT1 as assessed using whole-cell electrophysiology (such as described in Milligan et al. (2015) Ann Neurol. 75(4): 581-590; Barcia et al. (2012) Nat Genet. 44(11): 1255-1259; Mikati et al. (2015) Ann Neurol. 78(6): 995-999; or Rizzo et al. Mol Cell Neurosci. (2016) 72:54-63). This increase of whole cell current can be, for example, an increase of at least or about 50%, 100%, 150%, 200%, 250%, 300%, 350%, 400% or more. The subject can then be confirmed to have a disease or condition associated with a gain-of-function mutation in KCNT1.

In particular examples, the subject is confirmed as having a KCNT1 allele containing a gain-of-function mutation (e.g. V271F, G288S, R398Q, R428Q, R474Q, R474H, R474C, G652V, I760M, Y796H, M896I, P924L, R928C or A934T).

The compounds disclosed herein, or pharmaceutically acceptable salts, solvates or isotopologues thereof, or the pharmaceutical composition disclosed herein, can also be used therapeutically for conditions associated with excessive neuronal excitability where the excessive neuronal excitability is not necessarily the result of a gain-of-function mutation in KCNT1. Even in instances where the disease is not the result of increased KCNT1 expression and/or activity, inhibition of KCNT1 expression and/or activity can nonetheless result in a reduction in neuronal excitability, thereby providing a therapeutic effect. Thus, the compounds disclosed herein, or pharmaceutically acceptable salts, solvates or isotopologues thereof, or the pharmaceutical composition disclosed herein, can be used to treat a subject with conditions associated with excessive neuronal excitability, for example, epilepsy and other encephalopathies (e.g., epilepsy of infancy with migrating focal seizures (EIMFS), autosomal dominant nocturnal frontal lobe epilepsy (ADNFLE), West syndrome, infantile spasms, epileptic encephalopathy, focal epilepsy, Ohtahara syndrome, developmental and epileptic encephalopathy, Lennox Gastaut syndrome, Dravet syndrome, and seizures) or cardiac dysfunctions (e.g., cardiac arrhythmia, Brugada syndrome, myocardial infarction), regardless of whether or not the disease or disorder is associated with a gain-of-function mutation in KCNT1.

### Pharmaceutical Compositions and Routes of Administration

Compounds, and pharmaceutically acceptable salts, solvates or isotopologues thereof provided in accordance with the present invention, are usually administered in the form of pharmaceutical compositions. This invention therefore provides pharmaceutical compositions that contain, as the active ingredient, one or more of the compounds described, or a pharmaceutically acceptable salt, solvate or isotopologue thereof, and one or more pharmaceutically acceptable excipients, carriers, including inert solid diluents and fillers, diluents, including sterile aqueous solution and various organic solvents, permeation enhancers, solubilizers and adjuvants. The pharmaceutical compositions may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.)

The pharmaceutical compositions may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One mode for administration is parenteral, particularly by injection. The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present invention. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating a compound according to the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral administration is another route for administration of compounds in accordance with the invention. Administration may be via capsule or enteric coated tablets, or the like. In making the pharmaceutical compositions that include at least one compound described herein, the active ingredient is usually diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl and propylhydroxy-benzoates; sweetening agents; and flavoring agents.

The compositions of the invention can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. Preferably, for oral administration, each dosage unit contains from 1 mg to 2 g of a compound described herein, and for parenteral administration, preferably from 0.1 to 700 mg of a compound described herein. It will be understood, however, that the amount of the compound actually administered usually will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the compound of the present invention is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably, the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

The pharmaceutical composition may comprise a disclosed compound, or pharmaceutically acceptable salt, solvate or isotopologue thereof, and a pharmaceutically acceptable carrier.

The compounds of the present invention are inhibitors of KCNT1 activity. The Examples demonstrate that the compounds of the present invention provide potent in vitro KCNT1-inhibitory activity. For example, when tested in a KCNT1 patch clamp assay, the compounds may exhibit a pIC₅₀ of greater than 6 against each of wild-type and A934T, F346L, G288S variant KCNT1. The compounds exhibit a pIC₅₀ of greater than 7 against wild-type KCNT1. In a thallium flux assay, the compounds demonstrate a pIC₅₀ of 7 or more against A934T variant KCNT1.

The compounds of the present invention also exhibit low clearance by metabolic mechanisms. Thus, the compounds of the present invention provide a high level of in vivo stability. The Examples demonstrate that the compounds of the present invention provide low levels of in vitro human liver microsomal clearance and in vitro human hepatocyte clearance. For example, the compounds exhibit a clearance of less than 3 μL/min/mg.prot (BLQ) in a microsomal clearance assay and less than 4.5 μL/min/mg.prot in a hepatocyte clearance assay.

The compounds of the present invention also demonstrate solubility in biologically media relevant for oral administration, and adsorption in the intestine. The Examples demonstrate that the compounds of the present invention provide solubility in a fasted state simulated intestinal fluid solubility (FaSSIF) assay. For example, the compounds of the present invention demonstrate solubility in FaSSIF media of more than 40 μM.

The compounds of the present invention also indicate an ability to penetrate the central nervous system (CNS), as they show low efflux by Pgp (MDR1), one of the major transporters present in the blood-brain barrier (BBB), as assessed by MDCK-MDR1 permeability and efflux ratio (ER). The Examples demonstrate that the compounds of the present invention provide permeability in and low efflux ratio (ER) in the MDCK-MDR1 assay.

### EXAMPLES

In order that the invention described herein may be more fully understood, the following examples are set forth. The synthetic and biological examples described in this application are offered to illustrate the compounds, pharmaceutical compositions and methods provided herein and are not to be construed in any way as limiting their scope.

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimal reaction conditions may vary with the particular reactants or solvent used, but such conditions can be determined by one skilled in the art by routine optimization.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include recrystallization, filtration, flash chromatography, trituration, high pressure liquid chromatography (HPLC), or supercritical fluid chromatography (SFC). Note that flash chromatography may either be performed manually or via an automated system. The compounds provided herein may be characterized by known standard procedures, such as nuclear magnetic resonance spectroscopy (NMR) or liquid chromatography mass spectrometry (LCMS). NMR chemical shifts are reported in part per million (ppm) and are generated using methods well known to those of skill in the art.

### List of Abbreviations

° C.: degrees centigrade
μL: microlitres
BEH: Ethylene Bridged Hybrid
BLQ: Below the Limit of Quantification
CH₃CN: Acetonitrile
Clint: intrinsic Clearance
DCM: Dichloromethane
DIPEA: Diisopropylethylamine
DMF: Dimethylformamide
DMSO: Dimethylsulfoxide
EGTA: Ethylene glycol-bis(β-aminoethyl ether)-N,N,N′,N′-tetraacetic acid
ER: Efflux ratio
EtOAc: Ethyl acetate
EtOH: Ethanol
FaSSIF: Fasted state simulated intestinal fluid
HATU: 1-[Bis(dimethylamino)methylene]-1H-1,2,3-triazolo[4,5-b]pyridinium 3-oxide
hexafluorophosphate
HBSS: Hank's balanced salt solution
HEPES: (4-(2-hydroxyethyl)-1-piperazineethanesulfonic acid)
hr: Hour
HRMS: High Resolution Mass Spectrometry
hrs: Hours
IPA: Isopropanol
LCMS: Liquid chromatography mass spectrometry
m/z: mass to charge ratio
MDCK-MDR1: Madin-Darby canine kidney-multidrug resistance protein 1
MeOD: Deuteromethanol
MeOH: Methanol
min: Minutes
mL: millilitres
MS: Mass Spectrometry
n: number of replicates
NADPH: Nicotinamide adenine dinucleotide phosphate,
NMDG: N-Methyl-D-glucamine
NMR: Nuclear Magnetic Resonance
PDA: Photodiode Array
QDA: Quadrupole Dalton
r.t.: room temperature
SD: Standard Deviation
SE Clint: standard error intrinsic clearance
SFC: Super critical fluid chromatography
t_{1/2}: half life
T3P®: Propanephosphonic acid anhydride
TEER: Trans-epithelial electrical resistance
TFA: Trifluoroacetic acid
THF: Tetrahydrofuran
UPC2: Ultraperformance Convergence Chromatography
UPLC: Ultra Performance Liquid Chromatography
WT: Wild Type
   Example and Intermediate Compound Names Generated Using Biovia Draw 2024 Version 24.1 NET (64 Bit) from Dassault Systemes

### Synthesis of Intermediates

### Intermediate 1: Ethyl 3-(difluoromethyl)-1H-pyrazole-5-carboxylate

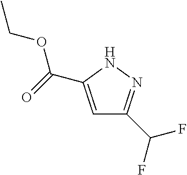

To a solution of 2,2-difluoroethylamine (4.35 mL, 61.7 mmol) in chloroform (150 mL) was added tert-butyl nitrite (8.87 mL, 74.0 mmol) and acetic acid (741 mg, 12.3 mmol). The reaction mixture was stirred at 80° C. for 60 min then cooled to r.t. Ethyl propiolate (3.18 mL, 30.8 mmol) was then added to the reaction mixture and the mixture was stirred at r.t. for 16 hrs. The mixture was concentrated under reduced pressure. The residue was diluted with DCM (100 mL), washed with a saturated aqueous NaHCO₃ solution (100 mL) and brine (100 mL), dried over MgSO₄, filtered and concentrated under reduced pressure to give the crude product. The crude product was purified by flash column chromatography on silica gel (24 g cartridge, 0-100% EtOAc in iso-hexane) to afford the title compound (1.3 g, 6.49 mmol, yield: 21%) as a pale-yellow oil which solidified on standing. ¹H NMR (400 MHz, DMSO) δ 14.46 (s, 1H), 7.22-6.87 (m, 2H), 4.33 (q, J=7.1 Hz, 2H), 1.31 (t, J=7.1 Hz, 3H). ¹⁹F NMR (376 MHz, DMSO) δ −111.35.

### Intermediate 2: Ethyl 2-(2,2-difluoroethyl)-5-(difluoromethyl)pyrazole-3-carboxylate

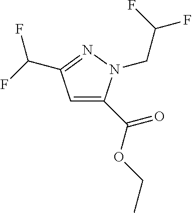 [SMILES: CCOC(=O)c1cc(C(F)F)nn1CC(F)F]

To a solution of Intermediate 1 (1.30 g, 6.49 mmol) and cesium carbonate (4.23 g, 13.0 mmol) in CH₃CN (20 mL) was added 2,2-difluoroethyl trifluoromethanesulfonate (95.0%, 1.82 mL, 13.0 mmol). The reaction mixture was stirred at r.t. for 2 hrs. The mixture was concentrated under reduced pressure then diluted with saturated aqueous NH₄Cl solution (50 mL). The mixture was extracted with EtOAc (50 mL×2). The combined organic phases were washed with water (50 mL) and brine (50 mL), dried over Na₂SO₄, filtered and concentrated under reduced pressure. The residue was purified by flash chromatography on silica gel (24 g cartridge, 0-60% EtOAc in heptane) to afford the title compound (1.45 g, 5.59 mmol, yield: 86%) as a colourless oil. ¹H NMR (400 MHz, DMSO) δ 7.20 (d, J=1.0 Hz, 1H), 7.09 (t, J=54.1 Hz, 1H), 6.44 (tt, J=54.8, 3.7 Hz, 1H), 5.03 (td, J=14.6, 3.7 Hz, 2H), 4.33 (q, J=7.1 Hz, 2H), 1.32 (t, J=7.1 Hz, 3H). ¹⁹F NMR (376 MHz, DMSO) δ −112.47, −122.60. LCMS (Waters Cortecs C18, 30×2.1 mm, 2.7 m, 90 Å at 40° C., 3.0 min method, 0.1% Formic acid in water, 2-100% CH₃CN/water): m/z 255.0 (M+H)+ (ES+) at 1.43 min, >99% purity 260+/−80 nm.

### Intermediate 3: 2-(2,2-Difluoroethyl)-5-(difluoromethyl)pyrazole-3-carboxylic acid

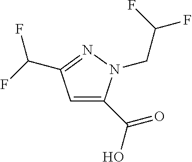 [SMILES: O=C(O)c1cc(C(F)F)nn1CC(F)F]

To a solution of Intermediate 2 (1.45 g, 5.59 mmol) in THF (20 mL) and water (10 mL) was added lithium hydroxide monohydrate (704 mg, 16.8 mmol). The mixture was stirred at r.t. for 1 hr. The mixture was concentrated under reduced pressure and dissolved in EtOAc (100 mL), washed with aqueous 1 N HCl (20 mL) solution, water (50 mL), dried over anhydrous MgSO₄, filtered and evaporated under reduced pressure to afford the title compound (1.25 g, 5.36 mmol, yield: 96%) as an off-white solid. ¹H NMR (400 MHz, DMSO) δ 13.98 (s, 1H), 7.13 (d, J=1.0 Hz, 1H), 7.07 (t, J=54.2 Hz, 1H), 6.42 (tt, J=54.9, 3.8 Hz, 1H), 5.03 (td, J=14.6, 3.8 Hz, 2H). ¹⁹F NMR (376 MHz, DMSO) δ −112.30, −122.53. LCMS (Water Cortecs C18, 90 Å, 30×2.1 mm, 2.7 m, at 40° C., 3 min method, 0.1% Formic acid in water, 2-100% CH₃CN/water): m/z 227.0 (M+H)⁺ (ES+); at 0.94 min, >97% purity at 260+/−80 nm.

### Intermediate 4: N′-hydroxy-2-methoxy-pyridine-4-carboxamidine

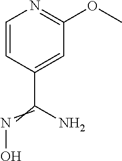

In a 250 mL round-bottomed flask was added 2-methoxyisonicotinonitrile (5.00 g, 37.3 mmol), hydroxylamine hydrochloride (3.89 g, 55.9 mmol) and sodium hydrogen carbonate (4.70 g, 55.9 mmol) followed by EtOH (60 mL). The slurry was heated to 80° C. and stirred for 2 hrs until full consumption of starting material was observed. The mixture was allowed to cool down to r.t. and concentrated under reduced pressure. The residue was resuspended in water (50 mL). The suspension was filtered, washed with extra water (˜100 mL) and dried under vacuum to afford the title compound (4.66 g, 27.6 mmol, yield: 74%) as a beige powder. ¹H NMR (500 MHz, DMSO) δ 10.01 (s, 1H), 8.13 (d, J=5.4 Hz, 1H), 7.25 (dd, J=5.4, 1.5 Hz, 1H), 7.07 (d, J=1.5 Hz, 1H), 5.96 (s, 2H), 3.85 (s, 3H).

### Intermediate 5: tert-Butyl N-[(1S)-1-[3-(2-methoxy-4-pyridyl)-1,2,4-oxadiazol-5-yl]ethyl]carbamate

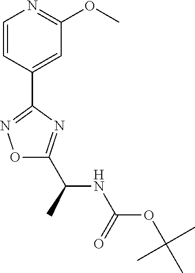 [SMILES: COc1cc(-c2noc([C@H](C)NC(=O)OC(C)(C)C)n2)ccn1]

To a solution of Intermediate 4 (1.00 g, 5.92 mmol), (tert-butoxycarbonyl)-L-alanine (98.0%, 1.37 g, 7.11 mmol) and HATU (3.38 g, 8.88 mmol) in dry DMF (10 mL) was added DIPEA (1.83 mL, 10.7 mmol) and the resulting solution was stirred at 70° C. for 18 hrs and then at r.t. for 48 hrs. The reaction mixture was diluted with EtOAc (50 mL) and washed with saturated aqueous NH₄Cl solution (2×50 mL). The aqueous layer was extracted with EtOAc (2×30 mL). The combined organic extracts were washed with saturated aqueous NaHCO₃ solution (2×100 mL) and brine (100 mL), then dried over Na₂SO₄, filtered, and concentrated under reduced pressure. The crude product was purified by flash chromatography on silica gel (25 g cartridge, 0-50% EtOAc in iso-hexane) to afford the title compound (1.07 g, 3.17 mmol, yield: 54%) as an off white solid. ¹H NMR (500 MHz, DMSO) δ 8.39 (d, J=5.3 Hz, 1H), 7.80 (d, J=7.5 Hz, 1H), 7.51 (dd, J=5.2, 1.4 Hz, 1H), 7.30-7.27 (m, 1H), 5.04-4.95 (m, 1H), 3.92 (s, 3H), 1.51 (d, J=7.1 Hz, 3H), 1.45-1.18 (m, 9H) (Boc appears rotameric). UPLC (BEH C18 Column at 40° C., 130 Å, 1.7 μm, 2.1 mm×30 mm, 3 min method, 0.1% Ammonium Hydroxide, 2-100% CH₃CN/water): m/z 321.3 (M+H)⁺ (ES+), at 1.51 min, 96.6% purity at 210-400 nm. Chiral analysis: The sample was dissolved in MeOH to 1 mg/mL and screened by SFC (Waters UPC2, with a PDA and a QDA detector). The optimal conditions are using a Chiralpak IH column, 4.6×250 mm, 5 μm, with mobile phase 20% IPA (0.1% Ammonia), 80% CO₂. The flow rate was 4 mL/min. The run time was 6.0 mins. The sample had an ee of >99% at 1.74 min.

### Intermediate 6: (1S)-1-[3-(2-Methoxy-4-pyridyl)-1,2,4-oxadiazol-5-yl]ethanamine

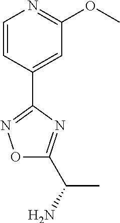 [SMILES: COc1cc(-c2noc([C@H](C)N)n2)ccn1]

TFA (4.84 mL, 63.2 mmol) was added to Intermediate 5 (1.06 g, 3.16 mmol) in DCM (10 mL) and the mixture was stirred for 2 hrs at r.t. The solvent was removed under reduced pressure and the residue was co-evaporated with toluene (2×20 mL). The residue was dissolved in EtOAc (50 mL), washed with saturated aqueous NaHCO₃ solution (50 mL). The aqueous layer was extracted with EtOAc (5×50 mL). The combined organic extracts were dried over Na₂SO₄, filtered and concentrated under reduced pressure to afford the title compound (668 mg, 2.88 mmol, yield: 91%) as an off white solid. ¹H NMR (400 MHz, DMSO) δ 8.38 (dd, J=5.3, 0.8 Hz, 1H), 7.52 (dd, J=5.3, 1.4 Hz, 1H), 7.30 (dd, J=1.4, 0.8 Hz, 1H), 4.30 (q, J=6.9 Hz, 1H), 3.92 (s, 3H), 2.30 (s, 2H), 1.45 (d, J=6.9 Hz, 3H). UPLC (BEH 18 Column at 40° C., 130 Å, 1.7 μm, 2.1 mm×30 mm, 3 min method, 0.1% Ammonium Hydroxide, 2-100% CH₃CN/water): m/z 221.3 (M+H)⁺ (ES+), at 0.91 min, 97% purity at 210-400 nm.

### Intermediate 7: 2-(Trideuteriomethoxy)pyridine-4-carbonitrile

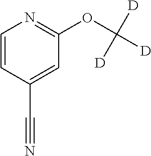 [SMILES: [2H]C([2H])([2H])Oc1cc(C#N)ccn1]

To a suspension of sodium hydride (60% in paraffin oil) (2.02 g, 50.5 mmol) in dry THF (60 mL) at 0° C. (ice bath) under nitrogen was added methanol-D₄ (2.20 mL, 54.1 mmol) dropwise. After stirring for 30 min, a solution of 2-chloropyridine-4-carbonitrile (5.00 g, 36.1 mmol) in dry THF (20 mL) was added dropwise. The reaction mixture was slowly warmed to r.t. and stirred for 16 h. The reaction mixture was cooled to 0° C. and diluted with ice-water (50 mL). The mixture was extracted with EtOAc (2×100 mL). The organic layer was washed with brine (100 mL), dried over anhydrous MgSO₄, filtered and concentrated in vacuo. The crude product was purified by flash chromatography on silica gel (80 g cartridge, 0-30% EtOAc/Iso-Hexane) to afford the title compound (3.91 g, 28.2 mmol, yield: 78.2%) as a white solid. ¹H NMR (400 MHz, DMSO-d6) δ 8.41 (dd, J=5.0, 1.0 Hz, 1H), 7.42-7.37 (m, 2H). LCMS (Water Cortecs C18, 90 Å, 30×2.1 mm, 2.7 m, at 40° C., 3 min method, 0.1% Formic acid in water, 2-100% MeCN/water): no mass, at 0.85 min, >99% purity at 260 nm+/−80 nm.

### Intermediate 8: N′-Hydroxy-2-(trideuteriomethoxy)pyridine-4-carboxamidine

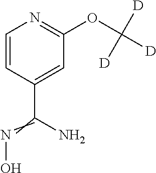 [SMILES: [2H]C([2H])([2H])Oc1cc(C(N)=NO)ccn1]

The title compound was prepared from Intermediate 7 (3.91 g, 28.2 mmol) in a corresponding fashion to Intermediate 4 as a white solid (3.55 g, 20.7 mmol, yield: 73.2%). ¹H NMR (400 MHz, DMSO-d6) δ 10.01 (s, 1H), 8.12 (d, J=5.4 Hz, 1H), 7.28-7.22 (m, 1H), 7.07 (dd, J=1.5, 0.7 Hz, 1H), 5.96 (s, 2H). UPLC (Waters ACQUITY UPLC® BEH C18, 1.7 μm, 2.1×30 mm at 40° C., 3 min method, 0.2% Ammonia in water, 2-100% CH₃CN/water): m/z 171.2 (M+H)+ (ES+), at 0.48 min, 91% purity at 260 nm+/−80 nm.

### Intermediate 9: tert-Butyl N-[(1S)-1-[3-[2-(trideuteriomethoxy)-4-pyridyl]-1,2,4-oxadiazol-5-yl]ethyl]carbamate

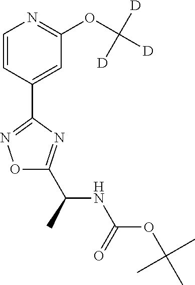 [SMILES: [2H]C([2H])([2H])Oc1cc(-c2noc([C@H](C)NC(=O)OC(C)(C)C)n2)ccn1]

The title compound was prepared from Intermediate 8 (1.00 g, 5.82 mmol) in a corresponding fashion to Intermediate 5 as a pale-yellow solid (1.02 g, 3.00 mmol, yield: 51.5%). ¹H NMR (400 MHz, DMSOd6) δ 8.39 (d, J=5.3 Hz, 1H), 7.80 (d, J=7.5 Hz, 1H), 7.51 (dd, J=5.3, 1.3 Hz, 1H), 7.33-7.24 (m, 1H), 5.07-4.91 (m, 1H), 1.51 (d, J=7.1 Hz, 3H), 1.40 (s, 9H). LCMS (Water Cortecs C18, 90 Å, 30×2.1 mm, 2.7 μm, at 40° C., 3 min method, 0.1% Formic acid in water, 2-100% MeCN/water): m/z 324.2 (M+H)⁺ (ES+), at 1.51 min, >99% purity at 260 nm+/−80 nm. Chiral analysis: The sample was dissolved in MeOH to 1 mg/mL and screened by SFC (Waters UPC2, with a PDA and a QDA detector). The optimal conditions are using a Phenomenex iA1 column, 4.6×250 mm, 5 μm, with mobile phase 15% MeOH (0.1% Ammonia), 85% CO₂. The flow rate was 4 mL/min. The run time was 2.0 mins. The sample had an ee of >99% at 1.32 min.

### Intermediate 10: (1S)-1-[3-[2-(Trideuteriomethoxy)-4-pyridyl]-1,2,4-oxadiazol-5-yl]ethanamine

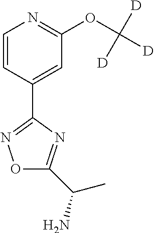 [SMILES: [2H]C([2H])([2H])Oc1cc(-c2noc([C@H](C)N)n2)ccn1]

The title compound was prepared from Intermediate 9 (404 mg, 1.19 mmol) in a corresponding fashion to Intermediate 6 as a pale-yellow oil (168 mg, 0.715 mmol, yield: 58%). ¹H NMR (500 MHz, MeOD) δ 8.35-8.18 (m, 1H), 7.59-7.46 (m, 1H), 7.44-7.31 (m, 1H), 4.42-4.29 (m, 1H), 1.62-1.54 (m, 3H). 2 exchangeable protons not observed. LCMS (Cortecs C18+, 90 Å at 40° C., 2.7 μm, 2.1 mm×30 mm, 1 min method, 0.1% formic acid, 2-100% CH₃CN/water): m/z 224.2 (M+H)⁺ (ES+) at 0.17 min, 100% purity at 254 nm.

### Example 1. 2-(2,2-Difluoroethyl)-5-(difluoromethyl)-N-[(1S)-1-[3-(2-methoxy-4-pyridyl)-1,2,4-oxadiazol-5-yl]ethyl]pyrazole-3-carboxamide (also known as (S)-1-(2,2-difluoroethyl)-3-(difluoromethyl)-N-(1-(3-(2-methoxypyridin-4-yl)-1,2,4-oxadiazol-5-yl)ethyl)-1H-pyrazole-5-carboxamide)

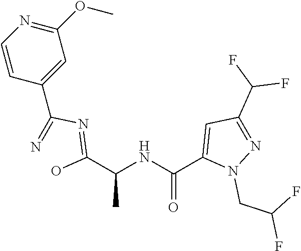 [SMILES: COc1cc(C(=N)/N=C(\O)[C@H](C)NC(=O)c2cc(C(F)F)nn2CC(F)F)ccn1]

To a solution of Intermediate 6 (0.667 g, 2.88 mmol), Intermediate 3 (716 mg, 3.16 mmol) and HATU (1.64 g, 4.32 mmol) in dry DMF (50 mL) was added DIPEA (1.50 mL, 8.63 mmol) and the resulting solution was stirred at r.t. for 18 hrs. The reaction mixture was diluted with EtOAc (100 mL) and washed with saturated aqueous NH₄Cl solution (2×100 mL), followed by saturated aqueous NaHCO₃ solution (2×100 mL), and brine (100 mL). The organic layer was dried over Na₂SO₄, filtered and concentrated under reduced pressure. The crude product was purified by flash chromatography on silica gel (40 g cartridge, 0-50% EtOAc in iso-hexane and then repurified by chromatography on silica gel (24 g cartridge, 0-50% tert-Butyl methyl ether in iso-hexane). The residue was dissolved in diethyl ether (10 mL) and precipitated with addition of iso-hexane (10 mL) and collected by filtration to afford the title compound (98%, 352 mg, 0.805 mmol, yield: 28%) as a fluffy white solid. The mixed fractions from both columns and the mother liquor from the precipitation were combined and the solvent removed under reduced pressure to afford a further crop of the title compound (0.748 g, 1.66 mmol, yield: 58%) as an off white solid. ¹H NMR (400 MHz, DMSO) δ 9.54 (d, J=7.2 Hz, 1H), 8.39 (dd, J=5.3, 0.8 Hz, 1H), 7.52 (dd, J=5.3, 1.4 Hz, 1H), 7.36-7.32 (m, 1H), 7.30 (dd, J=1.4, 0.8 Hz, 1H), 7.11 (t, J=54.3 Hz, 1H), 6.37 (tt, J=55.1, 3.6 Hz, 1H), 5.52-5.42 (m, 1H), 5.09-4.97 (m, 2H), 3.92 (s, 3H), 1.67 (d, J=7.1 Hz, 3H). ¹⁹F NMR (376 MHz, DMSO) δ −112.24, −122.54. UPLC (CSH C18 Column, 130 Å, 1.7 μm, 2.1 mm×30 mm, 10 min method, 0.1% Formic acid, 2-100% MeCN/water): m/z 429.3 (M+H)⁺ (ES+), at 4.22 min, 100% purity at 210-400 nm. Chiral analysis: The sample was dissolved in MeOH to 1 mg/mL and analysed by SFC (Waters UPC2, with a PDA and a QDA detector) using a Phenomenex C3 column, 4.6×250 mm, 5 μm, with mobile phase 15% MeOH (0.1% Ammonia), 85% C02. The flow rate was 4 mL/min. The run time was 2.0 mins. The sample had an ee of >99.9% at 1.13 min.

### Example 2. 2-(2,2-Difluoroethyl)-5-(difluoromethyl)-N-[(1S)-1-[3-[2-(trideuteriomethoxy)-4-pyridyl]-1,2,4-oxadiazol-5-yl]ethyl]pyrazole-3-carboxamide

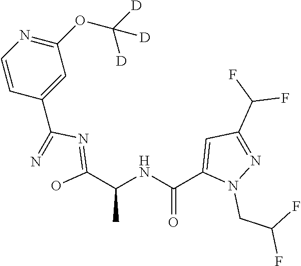 [SMILES: [2H]C([2H])([2H])Oc1cc(C(=N)/N=C(\O)[C@H](C)NC(=O)c2cc(C(F)F)nn2CC(F)F)ccn1]

To a mixture of Intermediate 10 (80 mg, 0.340 mmol), Intermediate 3 (81 mg, 0.340 mmol) and DIPEA (0.178 mL, 1.02 mmol) in EtOAc (2 mL) was added T3P® in EtOAc (50%, 0.301 mL, 0.511 mmol). The mixture was stirred at r.t. for 3 hrs. Additional DIPEA (0.0889 mL, 0.511 mmol) and T3P® in EtOAc (50%, 0.15 mL, 0.255 mmol) were added and the mixture stirred for 16 hrs. The reaction mixture was quenched with saturated aqueous NH₄Cl (75 mL) and EtOAc (40 mL) was added. The layers were separated. The organic layer was washed with saturated aqueous NaHCO₃ (75 mL), water (75 mL) and then brine (75 mL). The organic layer was dried over Na₂SO₄, filtered and concentrated under reduced pressure. The residue was purified by flash reverse phase chromatography (liquid load, 24 g cartridge, eluting 5-70% acetonitrile in water with 0.1% formic acid) to afford, after co-evaporation with toluene (10 mL), the title compound (107 mg, 0.248 mmol, yield: 73%) as an off-white solid. ¹H NMR (500 MHz, DMSO) δ 9.54 (d, J=7.2 Hz, 1H), 8.41-8.36 (m, 1H), 7.52 (dd, J=5.3, 1.4 Hz, 1H), 7.34 (s, 1H), 7.32-7.27 (m, 1H), 7.11 (t, J=54.3 Hz, 1H), 6.37 (tt, J=55.0, 3.7 Hz, 1H), 5.53-5.41 (m, 1H), 5.03 (td, J=14.7, 3.8 Hz, 2H), 1.67 (d, J=7.1 Hz, 3H). LCMS (Cortecs C18+, 90 Å at 40° C., 2.7 μm, 2.1 mm×30 mm, 3 min method, 0.1% formic acid, 2-100% CH₃CN/water): m/z 432.1 (M+H)⁺ (ES+) at 1.55 min, 100% purity at 254 nm+/−80 nm. Chiral analysis: The sample was dissolved in MeOH to 1 mg/mL and analysed by SFC (Waters UPC2, with a PDA and a QDA detector) using a Phenomenex C3 column, 4.6×250 mm, 5 μm, with mobile phase 15% MeOH (0.1% Ammonia), 85% CO₂. The flow rate was 4 mL/min. The run time was 2.0 min. The sample had an ee of 98.0% at 1.15 min.

### Biological Assays

### Efficacy of Exemplary Compounds in the Inhibition of KCNT1

### KCNT1—Patch Clamp Assay

Patch-clamp electrophysiological recording assays may be used to determine the potency (pIC₅₀) of test compounds to inhibit KCNT1 channels. Inhibition of KCNT1 was evaluated in inducible T-Rex Flp-In HEK293 cells stably expressing human WT or mutant A934T, G288S or F346L KCNT1, provided by Praxis Precision Medicines (Boston, US), in patch-clamp assays. Currents were recorded using the Qube 384 automated patch clamp system (Sophion). Pulse generation and data collection were performed Qube384 operation and analysis software (Sophion Viewpoint, Sophion Analyzer). Currents were recorded in perforated patch mode (10 μM escin) from a population of cells. The cells were treated with 1 μg/mL tetracycline at 16-24 h prior to assay to induce KCNT1 expression. On the day of the assay, cells were rinsed, lifted, triturated and resuspended at 2,000,000 cells/mL. The cells were allowed to recover onto a shaker at low speed prior to experimentation. Currents were recorded at 24° C. (±1° C.). The external solution contained the following (in mM): NaCl 105, NMDG 40, KCl 4, MgCl₂ 1, CaCl₂ 5, and HEPES 10 (pH=7.3-7.5, Osmolarity ˜300 mOsm). The extracellular solution was used as the wash, reference, and compound delivery solution. The internal solution contained the following (in mM): NaCl 70, KF 70, KCl 10, EGTA 5, HEPES 5, and Escin 0.01 (pH=7.1-7.3, Osmolarity ˜295 mOsm). The compounds were added in extracellular solution at 0.3% DMSO final concentration. Efficacy of compounds was measured on currents evoked by a 100 ms step from −80 m V to 0 mV. 100 μM Bepridil was used to completely inhibit KCNT1 current to allow for offline subtraction of non-KCNT1 current. The average mean current from 3 sweeps was calculated before and after compound addition, and the percent inhibition of each compound was calculated. The percent inhibition as a function of the compound concentration was fit from quadruplicate data using XLfit™ (IDBS, London, Great Britain) according to the following equation:

$$Y = Bottom + [ ( Top - Bottom ) / ( 1 + ( {( ( 1 {0}^{X} ) / ( {10}^{- pIC 50} ) )}^{nH} ) )$$

Where:
Bottom is data measured in presence of 100 μM bepridil compound in 0.3% DMSO
Top is data measured in 0.3% DMSO,
X is the concentration of test compound (log M),
pIC50 is the concentration of compound inhibiting by 50% of patch-clamp current (−log M),
nH is the Hill coefficient.
   pIC50s obtained in patch-clamp human KCNT1 assays are reported in the table below.

| | Example 1 | | | Example 2 | | |
|---|---|---|---|---|---|---|
| | pIC₅₀ | | | pIC₅₀ | | |
| KCNT1 variant | Mean | SD | n | Mean | SD | n |
| WT | 7.7 | 0.6 | 7 | 7.6 | 0.3 | 11 |
| A934T | 6.8 | 0.4 | 12 | 6.9 | 0.2 | 14 |
| F346L | 6.1 | 0.4 | 8 | 6.3 | 0.4 | 10 |
| G288S | 6.5 | 0.3 | 12 | 6.5 | 0.3 | 14 |

### Thallium Flux Assay

Thallium (Tl⁺) may be used as a surrogate of potassium to measure KCNT channel activity. The purpose of Tl⁺ based fluorescent assay is to determine the potency (pIC₅₀) of test compound to inhibit KCNT channel transport. Procedure is adapted from FluxOR Green Potassium Ion Channel Assay kit recommendations (Thermofisher). Briefly, inducible T-REx Flp-In HEK293 cells stably expressing human mutant A934T KCNT1 (UCB BioPharma SRL, Braine-l'Alleud, Belgium) are seeded in a microplate with culture medium containing 0.2 μg/ml doxycycline to induce KCNT channel expression for 24 hrs at 37° C. and 5% CO₂. Cells are incubated for one hr at r.t. in loading buffer containing fluorescent dye. Test compounds, in 0.5% DMSO, are then added for one hr preincubation at r.t. before baseline fluorescence is measured for 10 seconds. Intrinsic activity of mutant A934T KCNT1 channels is measured by adding 5 mM Tl⁺ and reading fluorescence signal for 2 min. Ratio of maximum fluorescence signal measured after Tl⁺ addition to baseline fluorescence signal (F_{MAX}/F_{Baseline}) and percent inhibition of Tl⁺ flux is calculated for each compound concentration. Data are analyzed by non-linear regression using XLfit™ (IDBS, London, Great Britain) according to the following equation:

$$Y = Bottom + [ ( Top - Bottom ) / ( 1 + ( {( ( 1 {0}^{X} ) / ( {10}^{- pIC 50} ) )}^{nH} ) )$$

Where:
Bottom is data measured in presence of 10 μM reference inhibitor compound in 0.5% DMSO
Top is data measured in 0.5% DMSO,
X is the concentration of test compound (log M),
pIC₅₀ is the concentration of test compound inhibiting by 50% of Tl+ flux (−log M), nH is the Hill coefficient.
   Reference inhibitor compound is tested in the same conditions to validate each A934T hKCNT1 experiment. pIC50 values obtained in Tl⁺ KCNT assays are reported below:

| | |
|---|---|
| Example 1 | Example 2 |
| pIC₅₀ | pIC₅₀ |

| Assay | Mean | SD | n | Mean | SD | n |
|---|---|---|---|---|---|---|
| Tl hKCNT1 A934T | 7.0 | 0.1 | 9 | 7.2 | 0.2 | 11 |

### Human Microsomal Clearance Assay

Human microsomal clearance assay may be used to determine the stability of the test compound (0.5 μM) in the presence of liver microsomes (1 mg/ml). Test compound is incubated over a 45-minute time course, analysed by FIRMS and the rate of parent disappearance is measured. Species specific liver microsomes, phosphate buffer/MgCl₂ and test compound are pre incubated at 37° C. for 5 min, prior to the addition of NADPH, to initiate the reaction. Each compound is incubated individually in a total incubation volume of 300 μL. A control incubation is included for each test compound where phosphate buffer/MgCl₂ is added instead of NADPH (minus co-factor). Reactions are terminated via temporal sampling at 0, 5, 15, 30 and 45 min, and 40 μL aliquot transferred into 120 μL ice-cold MeOH, on a cooler bed set to 4° C. Minus cofactor control is incubated for 45 min only. Following incubation, the termination plates are stored on cooler beds for ˜10 min until centrifugation at 3000 rpm for 10 min at 4° C. to precipitate the protein. CLint of the test compound and SE CLint, t_{1/2} and n (number of time points used to calculate CLint) are calculated. Percent remaining data for all samples is also assessed.

| | Human Microsomal Clearance |
|---|---|
| Example | (μL/min/mg · prot) |
| 1 | <3 (BLQ) |
| 2 | <3 (BLQ) |

### Human Hepatocyte Clearance Assay

Human hepatocyte clearance assay may be used to determine the stability of the test compound (at 0.5 μM) in the presence of cryopreserved hepatocytes (1×106 viable cells/mL). Test compound is incubated over a 120 min time course, analysed by FIRMS and the rate of parent disappearance is measured. Control compounds are also incubated under the same conditions to validate the assay. Suspension of cryopreserved hepatocytes (495 μL) pre-incubated for 5 min before addition of test compound (5 μL), with each compound incubated individually in a lid-covered plate for 0, 10, 20, 40, 60 and 120 min. Reactions are terminated via temporal sampling and transfer of 50 μL aliquot into 150 μL ice-cold methanol in a lid-covered plate. Termination plate stored at 4° C. during and for ˜10 min after incubation, then centrifuged at 2500 rpm at 4° C. for 30 min to precipitate the protein. CLint of the test compound and standard error (SE CLint), t_{1/2} and n (number of time points used to calculate CLint) are calculated.

| | Human Hepatocyte Clearance |
|---|---|
| Example | (μL/min/10⁶ cells) |
| 1 | 4.07 |
| 2 | <3 (BLQ) |

### Fasted State Simulated Intestinal Fluid Solubility (FaSSIF)

FaSSIF solubility values were determined using Biorelevant media (FaSSIF-V2) following manufacturer instructions for their preparation. The compound to be tested was added (as a dry powder) to media and incubated at 37° C. for 3 h. Filtered aliquots were analyzed on a Waters Xevo TQ-S UPLC-MS and quantified using Mass Lynx Software. For each compound, measurements were conducted in duplicate, and the average value was reported.

| Example | FaSSIF Solubility (μM) |
|---|---|
| 1 | 46 |
| 2 | 74 |

### MDCK-MDR1 Permeability (A-B or Bidirectional)

MDCK cells are an epithelial cell line of canine kidney origin. These cells can be stably transfected to express active human P glycoprotein (MDCK-MDR1) and are ideal for studying drug efflux. Test compound (2 μM) is added to either the apical or basolateral side of a confluent monolayer of MDCK-MDR1 cells and permeability is measured by monitoring the appearance of the test compound on the opposite side of the monolayer using LC MS/MS. If performing a bidirectional assay, the ER is calculated from the ratio of B-A and A-B permeabilities. On the day of the assay, TEER measurements are taken, then the monolayers are prepared by rinsing both apical and basolateral surfaces once with HBSS at the desired pH warmed to 37° C. Cells are then incubated at 37° C. in an atmosphere of 5% CO₂ and a relative humidity of 95% (without shaking), with the appropriate assay buffer in both apical (90 μl) and basolateral compartments (250 μl), for 30 min to stabilise physiological parameters. The fluorescent integrity marker lucifer yellow is also included in the apical dosing solution (A-B) and basolateral dosing solution (B-A). For assessment of A to B permeability, HBSS is removed from the apical compartment and replaced with test compound dosing solution. The apical compartment insert is then placed into a companion plate containing fresh buffer. For assessment of B to A permeability, HBSS is removed from the companion plate and replaced with test compound dosing solution. Fresh buffer is added to the apical compartment insert, which is then placed into the companion plate. At 60 min the apical compartment inserts, and the companion plates are separated, and apical and basolateral samples diluted for analysis. The integrity of the monolayer throughout the experiment is checked by monitoring lucifer yellow permeation using fluorometric analysis. Papp values are generated from A-B and B-A MDCK-MDR1 incubations along with experimental recoveries.

| Example | MDCK A-B/B-A (nms⁻¹) |
|---|---|
| 1 | 306/506 |
| 2 | 310/658 |

Furthermore, compounds of the present invention are subject to low efflux by Pgp (MDR1) and therefore show propensity to penetrate the CNS.

As discussed herein and demonstrated in the assays described above, the compounds of the present invention may provide potent inhibition of KCNT1, low levels of metabolic clearance, high in vivo stability and solubility in biological media, for example media relevant for oral administration, and intestinal permeability. In addition such compounds show propensity to penetrate the CNS.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the appended claims.

### Enumerated Embodiments

1. A compound of Formula I:

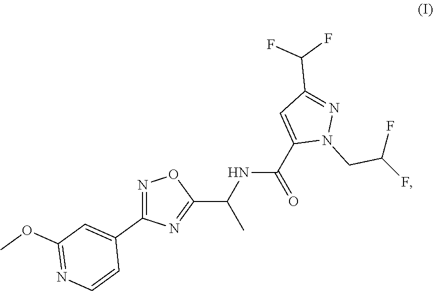 [SMILES: COc1cc(-c2noc(C(C)NC(=O)c3cc(C(F)F)nn3CC(F)F)n2)ccn1]

or a salt, solvate and/or isotopologue thereof.
2. The compound of embodiment 1 of Formula II:

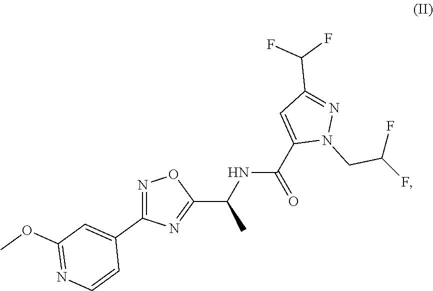 [SMILES: COc1cc(-c2noc([C@H](C)NC(=O)c3cc(C(F)F)nn3CC(F)F)n2)ccn1]

or a salt, solvate and/or isotopologue thereof.
3. The compound of embodiment 1, wherein the compound is:

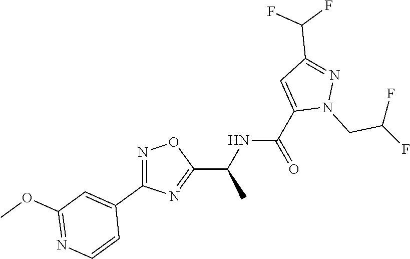 [SMILES: COc1cc(-c2noc([C@H](C)NC(=O)c3cc(C(F)F)nn3CC(F)F)n2)ccn1]

4. The compound of embodiment 1, wherein the compound is a salt of:

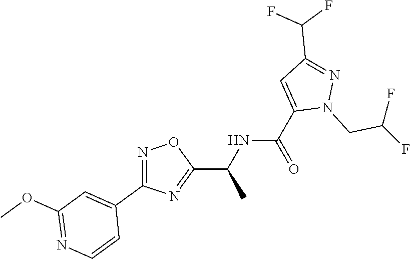 [SMILES: COc1cc(-c2noc([C@H](C)NC(=O)c3cc(C(F)F)nn3CC(F)F)n2)ccn1]

5. The compound of embodiment 4, wherein the compound is a pharmaceutically acceptable salt of:

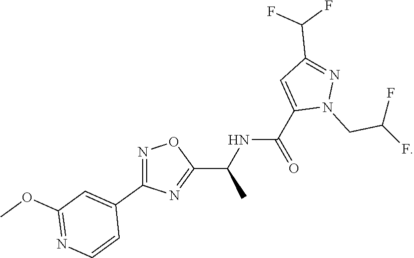 [SMILES: COc1cc(-c2noc([C@H](C)NC(=O)c3cc(C(F)F)nn3CC(F)F)n2)ccn1]

6. The compound of embodiment 1, wherein the compound is an isotopologue of:

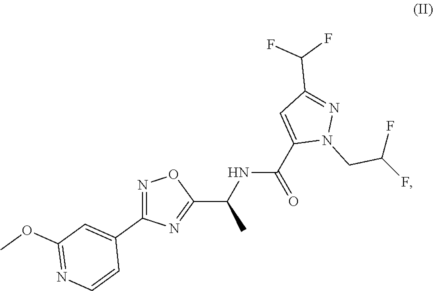 [SMILES: COc1cc(-c2noc([C@H](C)NC(=O)c3cc(C(F)F)nn3CC(F)F)n2)ccn1]

or a pharmaceutically acceptable salt and/or solvate thereof.
7. The compound of embodiment 6, wherein the compound is:

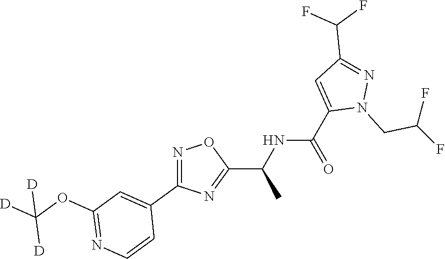 [SMILES: [2H]C([2H])([2H])Oc1cc(-c2noc([C@H](C)NC(=O)c3cc(C(F)F)nn3CC(F)F)n2)ccn1]

or a pharmaceutically acceptable salt and/or solvate thereof.
8. A pharmaceutical composition comprising the compound, or pharmaceutically acceptable salt, solvate or isotopologue thereof, of any one of embodiments 1 to 7, and a pharmaceutically acceptable excipient.
9. The compound, or pharmaceutically acceptable salt, solvate or isotopologue thereof, of any one of embodiments 1 to 7, or the pharmaceutical composition of embodiment 8, for use in therapy.
10. A method of treating a neurological disease or disorder, wherein the method comprises administering to a subject in need thereof the compound, or pharmaceutically acceptable salt, solvate or isotopologue thereof, of any one of embodiments 1 to 7 or the pharmaceutical composition of embodiment 8.
11. A method of treating a disease or condition associated with excessive neuronal excitability, wherein the method comprises administering to a subject in need thereof the compound, or pharmaceutically acceptable salt, solvate or isotopologue thereof, of any one of embodiments 1 to 7, or the pharmaceutical composition of embodiment 8.
12. A method of treating a disease or condition associated with a gain-of-function mutation of KCNT1, wherein the method comprises administering to a subject in need thereof the compound, or pharmaceutically acceptable salt, solvate or isotopologue thereof, of any one of embodiments 1 to 7, or the pharmaceutical composition of embodiment 8.
13. The compound, or pharmaceutically acceptable salt, solvate or isotopologue thereof, of any one of embodiments 1 to 7, or the pharmaceutical composition of embodiment 8, for use in treating a neurological disease or disorder.
14. The compound, or pharmaceutically acceptable salt, solvate or isotopologue thereof, of any one of embodiments 1 to 7, or the pharmaceutical composition of embodiment 8, for use in treating a disease or condition associated with excessive neuronal excitability.
15. The compound, or pharmaceutically acceptable salt, solvate or isotopologue thereof, of any one of embodiments 1 to 7, or the pharmaceutical composition of embodiment 8, for use in treating a disease or condition associated with a gain-of-function mutation of KCNT1.
16. The method of any one of embodiments 10 to 12, or the compound, or pharmaceutically acceptable salt, solvate or isotopologue thereof, or pharmaceutical composition, for use of any one of embodiments 13 to 15, wherein the neurological disease or disorder, the disease or condition associated with excessive neuronal excitability, or the disease or condition associated with a gain-of-function mutation of KCNT1 is a KCNT-1 related epilepsy or neurodevelopmental disorder.
17. The method of any one of embodiments 10 to 12, or the compound, or pharmaceutically acceptable salt, solvate or isotopologue thereof, or pharmaceutical composition, for use of any one of embodiments 13 to 15, wherein the neurological disease or disorder, the disease or condition associated with excessive neuronal excitability, or the disease or condition associated with a gain-of-function mutation of KCNT1 is epilepsy, an epilepsy syndrome, or an encephalopathy.
18. The method of any one of embodiments 10 to 12, or the compound, or pharmaceutically acceptable salt, solvate or isotopologue thereof, or pharmaceutical composition, for use of any one of embodiments 13 to 15, wherein the neurological disease or disorder, the disease or condition associated with excessive neuronal excitability, or the disease or condition associated with a gain-of-function mutation of KCNT1 is a genetic or pediatric epilepsy, genetic or pediatric epilepsy syndrome, or a genetic or pediatric developmental encephalopathy.
19. The method of any one of embodiments 10 to 12, or the compound, or pharmaceutically acceptable salt, solvate or isotopologue thereof, or pharmaceutical composition, for use of any one of embodiments 13 to 15, wherein the neurological disease or disorder, the disease or condition associated with excessive neuronal excitability, or the disease or condition associated with a gain-of-function mutation of KCNT1 is a cardiac dysfunction.
20. The method of any one of embodiments 10 to 12, or the compound, or pharmaceutically acceptable salt, solvate or isotopologue thereof, or pharmaceutical composition, for use of any one of embodiments 13 to 15, wherein the neurological disease or disorder, the disease or condition associated with excessive neuronal excitability, or the disease or condition associated with a gain-of-function mutation of KCNT1 is selected from the group consisting of epilepsy and other encephalopathies (e.g., epilepsy of infancy with migrating focal seizures (MMFSI, EIMFS), autosomal dominant nocturnal frontal lobe epilepsy (ADNFLE), West syndrome, infantile spasms, epileptic encephalopathy, focal epilepsy, Ohtahara syndrome, developmental and epileptic encephalopathy, Lennox Gastaut syndrome, Dravet syndrome, seizures (e.g., Generalized tonic clonic seizures, Asymmetric Tonic Seizures), leukodystrophy, leukoencephalopathy, intellectual disability, Multifocal Epilepsy, Drug resistant epilepsy, Temporal lobe epilepsy, or cerebellar ataxia).
21. The method of any one of embodiments 10 to 12, or the compound, or pharmaceutically acceptable salt, solvate or isotopologue thereof, or pharmaceutical composition, for use of any one of embodiments 13 to 15, wherein the neurological disease or disorder, the disease or condition associated with excessive neuronal excitability, or the disease or condition associated with a gain-of-function mutation of KCNT1 is selected from the group consisting of cardiac arrhythmia, sudden unexpected death in epilepsy, Brugada syndrome, and myocardial infarction.
22. The method of any one of embodiments 10 to 12, or the compound, or pharmaceutically acceptable salt, solvate or isotopologue thereof, or pharmaceutical composition, for use of any one of embodiments 13 to 15, wherein the neurological disease or disorder, the disease or condition associated with excessive neuronal excitability, or the disease or condition associated with a gain-of-function mutation of KCNT1 is selected from pain and related conditions (e.g. neuropathic pain, acute/chronic pain, migraine).
23. The method of any one of embodiments 10 to 12, or the compound, or pharmaceutically acceptable salt, solvate or isotopologue thereof, or pharmaceutical composition, for use of any one of embodiments 13 to 15, wherein the neurological disease or disorder, the disease or condition associated with excessive neuronal excitability, or the disease or condition associated with a gain-of-function mutation of KCNT1 is a muscle disorder (e.g. myotonia, neuromyotonia, cramp muscle spasms, spasticity).
24. The method of any one of embodiments 10 to 12, or the compound, or pharmaceutically acceptable salt, solvate or isotopologue thereof, or pharmaceutical composition, for use of any one of embodiments 13 to 15, wherein the neurological disease or disorder, the disease or condition associated with excessive neuronal excitability, or the disease or condition associated with a gain-of-function mutation of KCNT1 is selected from itch and pruritis, ataxia and cerebellar ataxias.
25. The method of any one of embodiments 10 to 12, or the compound, or pharmaceutically acceptable salt, solvate or isotopologue thereof, or pharmaceutical composition, for use of any one of embodiments 13 to 15, wherein the neurological disease or disorder, the disease or condition associated with excessive neuronal excitability, or the disease or condition associated with a gain-of-function mutation of KCNT1 is selected from psychiatric disorders (e.g. major depression, anxiety, bipolar disorder, schizophrenia).
26. The method of any one of embodiments 10 to 12, or the compound, or pharmaceutically acceptable salt, solvate or isotopologue thereof, or pharmaceutical composition, for use of any one of embodiments 13 to 15, wherein the neurological disease or disorder or the disease or condition associated with excessive neuronal excitability and/or a gain-of-function mutation in KCNT1 is selected from the group consisting of learning disorders, Fragile X, neuronal plasticity, and autism spectrum disorders.
27. The method of any one of embodiments 10 to 12, or the compound, or pharmaceutically acceptable salt, solvate or isotopologue thereof, or pharmaceutical composition, for use of any one of embodiments 13 to 15, wherein the neurological disease or disorder, the disease or condition associated with excessive neuronal excitability, or the disease or condition associated with a gain-of-function mutation of KCNT1 is selected from the group consisting of epileptic encephalopathy with SCN1A, SCN2A, SCN8A mutations, early infantile epileptic encephalopathy, Dravet syndrome, Dravet syndrome with SCN1A mutation, generalized epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, sudden unexpected death in epilepsy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, KCNT1 epileptic encephalopathy, early-onset epilepsy (EOE) and early-onset epileptic encephalopathy (EOEE).
28. A method of treating a KCNT-1 related epilepsy or neurodevelopmental disorder, wherein the method comprises administering to a subject in need thereof the compound, or pharmaceutically acceptable salt, solvate or isotopologue thereof, of any one of embodiments 1 to 7, or the pharmaceutical composition of embodiment 8.
29. The compound, or pharmaceutically acceptable salt, solvate or isotopologue thereof, of any one of embodiments 1 to 7, or the pharmaceutical composition of embodiment 8, for use in treating a KCNT-1 related epilepsy or neurodevelopmental disorder.

What is claimed is:

1. A compound of Formula I: 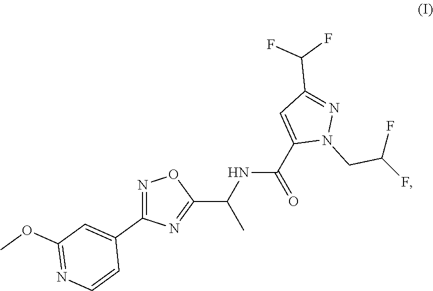 [SMILES: COc1cc(-c2noc(C(C)NC(=O)c3cc(C(F)F)nn3CC(F)F)n2)ccn1]
or a salt, solvate and/or isotopologue thereof.

2. The compound of claim 1 of Formula II: 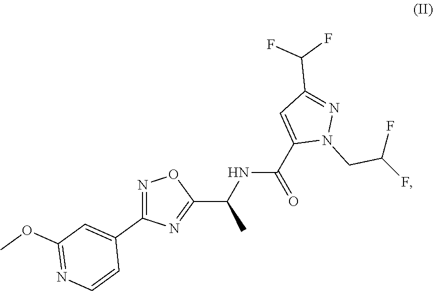 [SMILES: COc1cc(-c2noc([C@H](C)NC(=O)c3cc(C(F)F)nn3CC(F)F)n2)ccn1]
or a salt, solvate and/or isotopologue thereof.

3. The compound of claim 1, wherein the compound is:

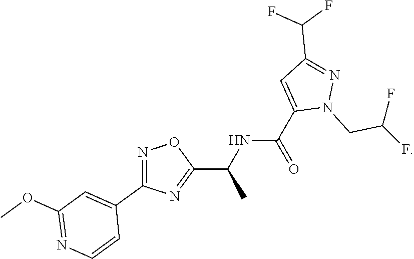 [SMILES: COc1cc(-c2noc([C@H](C)NC(=O)c3cc(C(F)F)nn3CC(F)F)n2)ccn1]

4. The compound of claim 1, wherein the compound is a salt of:

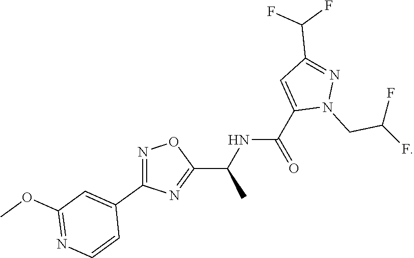 [SMILES: COc1cc(-c2noc([C@H](C)NC(=O)c3cc(C(F)F)nn3CC(F)F)n2)ccn1]

5. The compound of claim 4, wherein the compound is a pharmaceutically acceptable salt of:

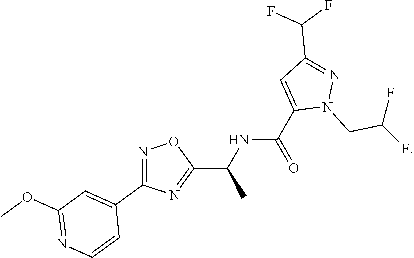 [SMILES: COc1cc(-c2noc([C@H](C)NC(=O)c3cc(C(F)F)nn3CC(F)F)n2)ccn1]

6. The compound of claim 1, wherein the compound is an isotopologue of: 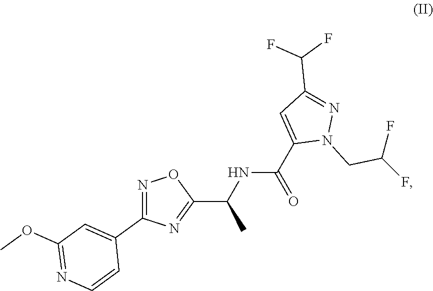 [SMILES: COc1cc(-c2noc([C@H](C)NC(=O)c3cc(C(F)F)nn3CC(F)F)n2)ccn1]
or a pharmaceutically acceptable salt and/or solvate thereof.

7. The compound of claim 6, wherein the compound is: 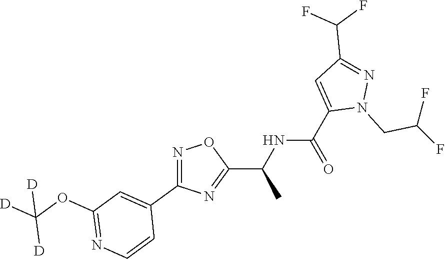 [SMILES: [2H]C([2H])([2H])Oc1cc(-c2noc([C@H](C)NC(=O)c3cc(C(F)F)nn3CC(F)F)n2)ccn1]
or a pharmaceutically acceptable salt and/or solvate thereof.

8. A pharmaceutical composition comprising the compound, or pharmaceutically acceptable salt, solvate or isotopologue thereof, of claim 1, and a pharmaceutically acceptable excipient.

9. A method of treating a neurological disease or disorder, wherein the method comprises administering to a subject in need thereof a compound according to claim 1, a pharmaceutically acceptable salt, solvate or isotopologue thereof, or a pharmaceutical composition comprising a compound according to claim 1 or a pharmaceutically acceptable salt, solvate or isotopologue thereof.

10. A method of treating a disease or condition associated with excessive neuronal excitability, wherein the method comprises administering to a subject in need thereof a compound according to claim 1, a pharmaceutically acceptable salt, solvate or isotopologue thereof, or a pharmaceutical composition comprising a compound according to claim 1 or a pharmaceutically acceptable salt, solvate or isotopologue thereof.

11. A method of treating a disease or condition associated with a gain-of-function mutation of KCNT1, wherein the method comprises administering to a subject in need thereof a compound according to claim 1, a pharmaceutically acceptable salt, solvate or isotopologue thereof, or a pharmaceutical composition comprising a compound according to claim 1 or a pharmaceutically acceptable salt, solvate or isotopologue thereof.

12. A method according to claim 9, wherein the neurological disease or disorder is a KCNT-1 related epilepsy or neurodevelopmental disorder.

13. A method according to claim 9, wherein the neurological disease or disorder is epilepsy, an epilepsy syndrome, or an encephalopathy.

14. A method according to claim 9, wherein the neurological disease or disorder is a genetic or pediatric epilepsy, genetic or pediatric epilepsy syndrome, or a genetic or pediatric developmental encephalopathy.

15. A method according to claim 9, wherein the neurological disease or disorder is selected from the group consisting of a cardiac dysfunction, epilepsy and other encephalopathies (e.g., epilepsy of infancy with migrating focal seizures (MMFSI, EIMFS), autosomal dominant nocturnal frontal lobe epilepsy (ADNFLE), West syndrome, infantile spasms, epileptic encephalopathy, focal epilepsy, Ohtahara syndrome, developmental and epileptic encephalopathy, Lennox Gastaut syndrome, Dravet syndrome, seizures (e.g., Generalized tonic clonic seizures, Asymmetric Tonic Seizures), leukodystrophy, leukoencephalopathy, intellectual disability, Multifocal Epilepsy, Drug resistant epilepsy, Temporal lobe epilepsy, cerebellar ataxia), cardiac arrhythmia, sudden unexpected death in epilepsy, Brugada syndrome, myocardial infarction pain and related conditions (e.g. neuropathic pain, acute/chronic pain, migraine), a muscle disorder (e.g. myotonia, neuromyotonia, cramp muscle spasms, spasticity), itch and pruritis, ataxia, cerebellar ataxias, psychiatric disorders (e.g. major depression, anxiety, bipolar disorder, schizophrenia), learning disorders, Fragile X, neuronal plasticity, autism spectrum disorders, epileptic encephalopathy with SCN1A, SCN2A, SCN8A mutations, early infantile epileptic encephalopathy, Dravet syndrome, Dravet syndrome with SCN1A mutation, generalized epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, sudden unexpected death in epilepsy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, KCNT1 epileptic encephalopathy, early-onset epilepsy (EOE) and early-onset epileptic encephalopathy (EOEE).
